SEQUENCE LISTING (46 sequences)

SEQ ID NO: 1 - protein, 12 aa, Artificial
  Synthetic
GSXHSXEXXRWK

SEQ ID NO: 2 - protein, 12 aa, Artificial
  Synthetic
GTXHSXEXXRWK

SEQ ID NO: 3 - protein, 12 aa, Artificial
  Synthetic
GQXHSXEXXRWK

SEQ ID NO: 4 - protein, 12 aa, Artificial
  Sythetic
GSXHSXEXXRWK

SEQ ID NO: 5 - protein, 12 aa, Artificial
  Synthetic
GTXHSXEXXRWK

SEQ ID NO: 6 - protein, 12 aa, Artificial
  Synthetic
GQXHSXEXXRWK

SEQ ID NO: 7 - protein, 12 aa, Artificial
  Synthetic
GSXHTXEXXRWK

SEQ ID NO: 8 - protein, 12 aa, Artificial
  Synthetic
GTXHTXEXXRWK

SEQ ID NO: 9 - protein, 12 aa, Artificial
  Synthetic
GQXHTXEXXRWK

SEQ ID NO: 10 - protein, 12 aa, Artificial
  Synthetic
GSXHTXEXXRWK

SEQ ID NO: 11 - protein, 12 aa, Artificial
  Synthetic
GTXHTXEXXRWK

SEQ ID NO: 12 - protein, 12 aa, Artificial
  Synthetic
GQXHTXEXXRWK

SEQ ID NO: 13 - protein, 12 aa, Artificial
  Synthetic
GSXHSXEXXRWK

SEQ ID NO: 14 - protein, 12 aa, Artificial
  Synthetic
GTXHSXEXXRWK

SEQ ID NO: 15 - protein, 12 aa, Artificial
  Synthetic
GQXHSXEXXRWK

SEQ ID NO: 16 - protein, 12 aa, Artificial
  Synthetic
GSXHSXEXXRWK

SEQ ID NO: 17 - protein, 12 aa, Artificial
  Synthetic
GTXHSXEXXRWK

SEQ ID NO: 18 - protein, 12 aa, Artificial
  Synthetic
GQXHSXEXXRWK

SEQ ID NO: 19 - protein, 12 aa, Artificial
  Synthetic
GSXHTXEXXRWK

SEQ ID NO: 20 - protein, 12 aa, Artificial
  Synthetic
GTXHTXEXXRWK

SEQ ID NO: 21 - protein, 12 aa, Artificial
  Synthetic
GQXHTXEXXRWK

SEQ ID NO: 22 - protein, 12 aa, Artificial
  Synthetic
GSXHTXEXXRWK

SEQ ID NO: 23 - protein, 12 aa, Artificial
  Synthetic
GTXHTXEXXRWK

SEQ ID NO: 24 - protein, 12 aa, Artificial
  Synthetic
GQXHTXEXXRWK

SEQ ID NO: 25 - protein, 12 aa, Artificial
  Synthetic
GSXHSXENXRWK

SEQ ID NO: 26 - protein, 12 aa, Artificial
  Synthetic
GTXHSXENXRWK

SEQ ID NO: 27 - protein, 12 aa, Artificial
  Synthetic
GQXHSXENXRWK

SEQ ID NO: 28 - protein, 12 aa, Artificial
  Synthetic
GSXHSXENXRWK

SEQ ID NO: 29 - protein, 12 aa, Artificial
  Synthetic
GTXHSXENXRWK

SEQ ID NO: 30 - protein, 12 aa, Artificial
  Synthetic
GQXHSXENXRWK

SEQ ID NO: 31 - protein, 12 aa, Artificial
  Synthetic
GSXHTXENXRWK

SEQ ID NO: 32 - protein, 12 aa, Artificial
  Synthetic
GTXHTXENXRWK

SEQ ID NO: 33 - protein, 12 aa, Artificial
  Synthetic
GQXHTXENXRWK

SEQ ID NO: 34 - protein, 12 aa, Artificial
  Synthetic
GSXHTXENXRWK

SEQ ID NO: 35 - protein, 12 aa, Artificial
  Synthetic
GTXHTXENXRWK

SEQ ID NO: 36 - protein, 12 aa, Artificial
  Synthetic
GQXHTXENXRWK

SEQ ID NO: 37 - protein, 12 aa, Artificial
  Synthetic
GSXHSXXXXRWX

SEQ ID NO: 38 - protein, 12 aa, Artificial
  Synthetic
GSXHXXCXXRWX

SEQ ID NO: 39 - protein, 12 aa, Artificial
  Synthetic
GSXHXXCXXRWC

SEQ ID NO: 40 - protein, 12 aa, Artificial
  Synthetic
GSXHSXEXXRWK

SEQ ID NO: 41 - protein, 12 aa, Artificial
  Synthetic
GEXHSXEXXRWK

SEQ ID NO: 42 - protein, 12 aa, Artificial
  Synthetic
ESXHEXEXXRWK

SEQ ID NO: 43 - protein, 12 aa, Artificial
  Synthetic
GSXHSAEXXRWK

SEQ ID NO: 44 - protein, 12 aa, Artificial
  Synthetic
GSXHSXEXXRWK

SEQ ID NO: 45 - protein, 12 aa, Artificial
  Synthetic
GSXHSXEQXRWK

SEQ ID NO: 46 - protein, 12 aa, Artificial
  Synthetic
GSXHSXEXXRWK

(19) United States
(12) Patent Application Publication
(10) Pub. No.: US 2008/0280820 A1
(43) Pub. Date: Nov. 13, 2008

(54) Novel Peptides for Use in the Treatment of Obesity

(76) Inventors: Ulrich Sensfuss, Kobenhavn V (DK); Leif Christensen, Roskilde (DK)

Correspondence Address: NOVO NORDISK, INC.;INTELLECTUAL PROPERTY DEPARTMENT, 100 COLLEGE ROAD WEST, PRINCETON, NJ, 08540, US

(21) Appl. No.: 11/666,795

(22) PCT Filed: Nov. 4, 2005
(86) PCT No.: PCT/EP2005/055762

§ 371 Date: Oct. 4, 2007

(30) Foreign Application Priority Data
Nov. 4, 2004 (PA) 2004 01696

(51) Int. Cl. A61K 38/12; C07K 7/64; A61P 3/10; A61P 3/04

(52) U.S. Cl. 514/11; 530/317

(57) ABSTRACT

The present invention relates to novel peptide compounds which are effective in modulating one or more melanocortin receptor types, to the use of the compounds in therapy, to methods of treatment comprising administration of the compounds to patients in need thereof, and to the use of the compounds in the manufacture of medicaments. The compounds of the invention are of particular interest in relation to the treatment of obesity as well as a variety of diseases or conditions associated with obesity.

### FIELD OF THE INVENTION

The present invention relates to novel peptide compounds which are ligands for one or more melanocortin receptors and which may exert prolonged activity, to the use of the compounds in therapy, to methods of treatment comprising administration of the compounds to patients, and to the use of the compounds in the manufacture of medicaments.

### BACKGROUND OF THE INVENTION

Obesity is a well known risk factor for the development of many very common diseases such as atherosclerosis, hypertension, type 2 diabetes (non-insulin dependent diabetes mellitus (NIDDM)), dyslipidaemia, coronary heart disease, and osteoarthritis and various malignancies. It also causes considerable problems through reduced motility and decreased quality of life. The incidence of obesity and thereby also these diseases is increasing throughout the entire industrialised world. Only a few pharmacological treatments are available to date, namely Sibutramine (Abbot; acting via serotonergic and noradrenaline mechanisms) and Orlistat (Roche Pharm; reducing fat uptake from the gut). However, due to the important effect of obesity as a risk factor in serious (and even fatal) and common diseases, there is still a need for pharmaceutical compounds useful in the treatment of obesity.

The term obesity implies an excess of adipose tissue. In this context, obesity is best viewed as any degree of excess adiposity that imparts a health risk. The distinction between normal and obese individuals can only be approximated, but the health risk imparted by obesity is probably a continuum with increasing adiposity. However, in the context of the present invention, individuals with a Body Mass Index (BMI=body weight in kilograms divided by the square of the height in meters) above 25 are to be regarded as obese.

Even mild obesity increases the risk for premature death, diabetes, hypertension, atherosclerosis, gallbladder disease and certain types of cancer. In the industrialized western world the prevalence of obesity has increased significantly in the past few decades. Because of the high prevalence of obesity and its health consequences, its treatment should be a high public health priority.

When energy intake exceeds energy expenditure, the excess calories are stored in adipose tissue, and if this net positive balance is prolonged, obesity results, i.e. there are two components to weight balance, and an abnormality on either side (intake or expenditure) can lead to obesity.

Pro-opiomelanocortin (POMC) is the precursor for β-endorphin and melanocortin peptides, including melanocyte stimulating hormone (α-MSH) and adrenocorticotropin (ACTH). POMC is expressed in several peripheral and central tissues including melanocytes, the pituitary, and neurons of the hypothalamus. The POMC precursor is processed differently in different tissues, resulting in the expression of different melanocortin peptides depending on the site of expression. In the anterior lobe of the pituitary, mainly ACTH is produced whereas in the intermediate lobe and the hypothalamic neurons the major peptides are A-MSH, β-MSH, desacetyl-α-MSH and β-endorphin. Several of the melanocortin peptides, including ACTH and α-MSH, have been demonstrated to have appetite-suppressing activity when administered to rats by intracerebroventricular injection [Vergoni et al, European Journal of Pharmacology 179, 347-355 (1990)]. An appetite-suppressing effect is also obtained with the artificial cyclic α-MSH analogue, MT-II.

A family of five melanocortin receptor subtypes has been identified (melanocortin receptor 1-5, also called MC1, MC2, MC3, MC4 and MC5). The MC1, MC2 and MC5 are mainly expressed in peripheral tissues, whereas MC3 and MC4 are mainly centrally expressed; MC3 are, however, also expressed in several peripheral tissues. In addition to being involved in energy homeostasis, MC3 receptors have also been suggested to be involved in several inflammatory diseases. An MC3 agonist could have a positive effect on such diseases, e.g. gouty arthritis. MC5 are mainly peripherally expressed, and have been suggested to be involved in exocrine secretion and in inflammation. MC4 have been shown to be involved in the regulation of body weight and feeding behaviour, as MC4 knock-out mice develop obesity [Huzar et al., Cell 88, 131-141 (1997)]. In addition, the MC4 receptor has been shown to be involved in the regulation of energy expenditure [Fekete et al., Journal of Neuroscience 20, 1550-1558 (2000)]. Furthermore, studies of either ectopic central expression of agouti protein (MC1, MC3 and MC4 antagonist) or over-expression of an endogenously occurring MC3 and MC4 antagonist (agouti gene related protein, AGRP) in mouse brain demonstrated that the over-expression of these two antagonists led to the development of obesity [Kleibig et al., PNAS 92, 4728-4732 (1995)]. Moreover, icv injection of a C-terminal fragment of AGRP increases feeding and antagonizes the inhibitory effect of α-MSH on food intake.

In humans, several cases of families with obesity which is presumably due to frame shift mutations in MC4 have been described [see, e.g., Yeo et al., Nature Genetics 20, 111-112 (1998); Vaisse et al., Nature Genetics 20, 113-114 (1998)].

In conclusion, a MC4 agonist could serve as an anorectic drug or energy expenditure regulating drug and be useful in the treatment of obesity or obesity-related diseases, as well as in the treatment of other diseases, disorders or conditions which may be ameliorated by activation of MC4.

MC4 antagonists may be useful for treatment of cachexia or anorexia, and for treatment of waisting in frail elderly patients. Furthermore, MC4 antagonists may be used for treatment of chronic pain, neuropathy and neurogenic inflammation.

A large number of patent applications disclose various classes of non-peptidic small molecules as melanocortin receptor modulators; examples hereof are WO 03/009850, WO 03/007949 and WO 02/081443.

The use of peptides as melanocortin receptor modulators is disclosed in a number of patent documents, e.g. WO 03/006620, U.S. Pat. No. 5,731,408 and WO 98/27113. Hadley [Pigment Cell Res., 4, 180-185, (1991)] reports a prolonged effect of specific melanotropic peptides conjugated to fatty acids, the prolongation being effected by a transformation of the modulators from being reversibly acting to being irreversibly acting caused by the conjugated fatty acids.

### SUMMARY OF THE INVENTION

The present inventors have surprisingly found that specific peptide conjugates have a high modulating effect on one or more melanocortin receptors, i.e. the MC1, MC2, MC3, MC4 or MC5. Accordingly, the invention relates to compounds of formula I:

R¹—S-Z¹-Z²-Z³-His-Z⁴-Z⁵-c[X¹—X²—X³-Arg-X⁴—X⁵]N(R′)₂ [I]

wherein R¹ represents a straight-chain, branched and/or cyclic C₁₄₋₂₂alkanoyl, C₁₄₋₂₂alkenoyl or C₁₄₋₂₂alkynoyl which may optionally be substituted with one or more substituents selected from halogen, hydroxy and aryl, or R¹ represents C₉₋₁₇—C(O)—NH—S(O)₂—(CH₂)₃—C(O)—;
S represents a bond, a 4-aminobutyric acid residue, Gly, β-Ala or a structure represented by formula II

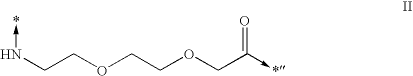

Z¹ represents Gly, β-Ala, Ser, D-Ser, Thr, D-Thr, His, D-His, Asn, D-Asn, Gln, D-Gln, Glu, D-Glu, Asp, D-Asp, Ala or D-Ala;
Z² represents Ser, Thr, Gln, Asn, Glu, Asp or His;
Z³ represents D-Gln or D-Asn;
Z⁴ represents Ser, Thr, Dab, Dap, Glu or Asp;
Z⁵ represents Ala, Val, Leu, Ile, Met or Nle;
X¹ represents Glu, Asp, Cys, homoCys, Pen, Lys, Orn, Dab or Dap;
X² represents His, Cit, Dab, Dap, Cgl, Cha, Val, Ile, tBuGly, Leu, Tyr, Glu, Ala, Nle, Met, Met(O), Met(O₂), Gln, Gln(alkyl), Gln(aryl), Asn, Asn(alkyl), Asn(aryl), Ser, Thr, Cys, Pro, Hyp, Tic, 2-PyAla, 3-PyAla, 4-PyAla, (2-thienyl)alanine, 3-(thienyl)alanine, (4-thiazolyl)Ala, (2-furyl)alanine, (3-furyl)alanine or Phe, wherein the phenyl moiety of said Phe is optionally substituted with a substituent selected among halogen, hydroxy, alkoxy, nitro, benzoyl, methyl, trifluoromethyl, amino and cyano;
X³ represents D-Phe, wherein the phenyl moiety in D-Phe may optionally be substituted with one or more substituents selected among halogen, hydroxy, alkoxy, nitro, methyl, trifluoromethyl and cyano;
X⁴ represents Trp, 2-Nal, a (3-benzo[b]thienyl)alanine residue or a (S)-2,3,4,9-tetrahydro-1H-β-carboline-3-carboxylic acid residue;
X⁵ represents Glu, Asp, Cys, homoCys, Pen, Lys, Orn, Dab or Dap;
wherein X¹ and X⁵ are joined, rendering the compound of formula I cyclic, either via a disulfide bridge deriving from X¹ and X⁵ both independently being Cys, homoCys or Pen, or via an amide bond formed between a carboxylic acid in the side-chain of X¹ and an amino group in the side chain of X⁵, or between a carboxylic acid in the side-chain of X⁵ and an amino group in the side-chain of X¹;
each R′ independently represents hydrogen or C₁₋₆alkyl, which may optionally be substituted with one or more amino or hydroxy;
with the proviso that the compound of formula I is not tetradecanoyl-Gly-Ser-D-Gln-His-Thr-Nle-c[Glu-Hyp-D-Phe-Arg-Trp-Lys]-NH₂, hexadecanoyl-Gly-Ser-D-Gln-His-Thr-Nle-c[Glu-Hyp-D-Phe-Arg-Trp-Lys]-NH₂, octadecanoyl-Gly-Ser-D-Gln-His-Thr-Nle-c[Glu-Hyp-D-Phe-Arg-Trp-Lys]-NH₂ or hexadecanoyl-Gly-Ser-D-Asn-His-Thr-Nle-c[Glu-Hyp-D-Phe-Arg-Trp-Lys]-NH₂;
and pharmaceutically acceptable salts, prodrugs and solvates thereof.

The invention further relates to the use of compounds of the invention in therapy, to pharmaceutical compositions comprising compounds of the invention, to methods of treatment comprising administration of compounds of the invention to patients in need thereof, and to the use of compounds of the invention in the manufacture of medicaments.

### DEFINITIONS

The use of a prefix of the type “C_{x-y}” preceding the name of a radical, such as in C_{x-y}alkyl (e.g. C₁₄₋₂₂alkyl) is intended to indicate a radical of the designated type having from x to y carbon atoms. Thus, straight-chain, branched and/or cyclic C₁₄₋₂₂alkanoyl, C₁₄₋₂₂alkenoyl or C₁₄₋₂₂alkynoyl groups as they occur as substituents R¹ in compounds of the present invention embrace straight-chain, branched and/or cyclic alkanoyl, alkenoyl or alkynoyl groups having 14, 15, 16, 17, 18, 19, 20, 21 or 22 carbon atoms (i.e. C₁₄, C₁₅, C₁₆, C₁₇, C₁₈, C₁₉, C₂₀, C₂₁ or C₂₂).

The term “alkyl” as used herein refers to a straight-chain, branched and/or cyclic, saturated monovalent hydrocarbon radical. Examples hereof include methyl, ethyl, 1-propyl, 2-propyl, 1-butyl, 2-butyl, tert-butyl, cyclopropyl, cyclobutyl, cyclopentyl and cyclohexyl.

The term “alkenyl” as used herein refers to a straight-chain, branched and/or cyclic, monovalent hydrocarbon radical comprising at least one carbon-carbon double bond. Examples hereof include ethenyl, prop-1-en-1-yl, prop-2-en-1-yl and prop-2-en-2-yl.

The term “alkynyl” as used herein refers to a straight-chain, branched and/or cyclic, monovalent hydrocarbon radical comprising at least one carbon-carbon triple bond, and it may optionally also comprise one or more carbon-carbon double bonds. Examples hereof include ethynyl, prop-1-yn-1-yl and prop-2-yn-1-yl.

The term “alkanoyl” as used herein is intended to indicate a radical of the formula —C(O)—R′, wherein R′ is alkyl as indicated above.

The term “alkenoyl” as used herein is intended to indicate a radical of the formula —C(O)—R″, wherein R″ is alkenyl as indicated above.

The term “alkynoyl” as used herein is intended to indicate a radical of the formula —C(O)—R′″, wherein R′″ is alkynyl as indicated above.

The term “alkoxy” as used herein is intended to indicate a radical of the formula —OR′, wherein R′ is alkyl as indicated above. Examples hereof include methoxy and ethoxy.

In the present context, the term “aryl” is intended to indicate a carbocyclic aromatic ring radical or a fused aromatic ring system radical wherein at least one of the rings is aromatic. Typical aryl groups include phenyl, biphenylyl, naphthyl, and the like.

The term “halogen” is intended to indicate members of the 7^{th} main group of the periodic table of the elements, which includes fluorine, chlorine, bromine and iodine (corresponding to fluoro, chloro, bromo and iodo substituents, respectively).

When two amino acids are said to be bridged, it is intended to indicate that functional groups in the side-chains of the two respective amino acids have reacted to form a covalent bond.

In the present context, the term “agonist” is intended to indicate a substance (ligand) that activates the receptor type in question.

In the present context, the term “antagonist” is intended to indicate a substance (ligand) that blocks, neutralizes or counteracts the effect of an agonist.

More specifically, receptor ligands may be classified as follows:

Receptor agonists, which activate the receptor; partial agonists also activate the receptor, but with lower efficacy than full agonists. A partial agonist will behave as a receptor partial antagonist, partially inhibiting the effect of a full agonist

Receptor neutral antagonists, which block the action of an agonist, but do not affect the receptor-constitutive activity.

Receptor inverse agonists, which block the action of an agonist and at the same time attenuate the receptor-constitutive activity. A full inverse agonist will attenuate the receptor-constitutive activity completely; a partial inverse agonist will attenuate the receptor-constitutive activity to a lesser extent.

As used herein the term “antagonist” includes neutral antagonists and partial antagonists, as well as inverse agonists. The term “agonist” includes full agonists as well as partial agonists.

In the present context, the term “pharmaceutically acceptable salt” is intended to indicate a salt which is not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric and nitric acids, and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene-salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. (1977) 66, 2, which is incorporated herein by reference. Examples of relevant metal salts include lithium, sodium, potassium and magnesium salts, and the like. Examples of alkylated ammonium salts include methylammonium, dimethylammonium, trimethylammonium, ethylammonium, hydroxyethylammonium, diethylammonium, butylammonium and tetramethylammonium salts, and the like.

As used herein, the term “therapeutically effective amount” of a compound refers to an amount sufficient to cure, alleviate or partially arrest the clinical manifestations of a given disease and/or its complications. An amount adequate to accomplish this is defined as a “therapeutically effective amount”. Effective amounts for each purpose will depend on the severity of the disease or injury, as well as on the weight and general state of the subject. It will be understood that determination of an appropriate dosage may be achieved using routine experimentation, by constructing a matrix of values and testing different points in the matrix, all of which is within the level of ordinary skill of a trained physician or veterinarian.

The terms “treatment”, “treating” and other variants thereof as used herein refer to the management and care of a patient for the purpose of combating a condition, such as a disease or a disorder. The terms are intended to include the full spectrum of treatments for a given condition from which the patient is suffering, such as administration of the active compound(s) in question to alleviate symptoms or complications thereof, to delay the progression of the disease, disorder or condition, to cure or eliminate the disease, disorder or condition, and/or to prevent the condition, in that prevention is to be understood as the management and care of a patient for the purpose of combating the disease, condition, or disorder, and includes the administration of the active compound(s) in question to prevent the onset of symptoms or complications. The patient to be treated is preferably a mammal, in particular a human being, but treatment of other animals, such as dogs, cats, cows, horses, sheep, goats or pigs, is within the scope of the invention.

As used herein, the term “solvate” refers to a complex of defined stoichiometry formed between a solute (in casu, a compound according to the present invention) and a solvent. Solvents may include, by way of example, water, ethanol, or acetic acid.

The amino acid abbreviations used in the present context have the following meanings:

| | |
|---|---|
| Ala | Alanine |
| Asn | Asparagines |
| Asn(alkyl) | 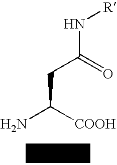 |
| Asn(aryl) | 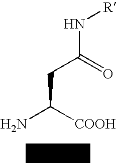 |
| Asp | aspartic acid |
| Arg | Arginine |
| β-Ala | 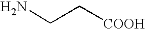 |
| Cha | 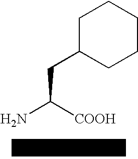 |
| Cgl | 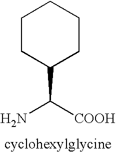 |
| Cit | Citrulline |
| Cys | Cysteine |
| Dab | (S)-2,4-diaminobutyric acid |
| Dap | (S)-2,3-diaminopropionic acid |
| D-Phe | 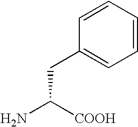 |
| Gln | Glutamine |
| Gln(alkyl) | 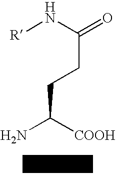 |
| Gln(aryl) | 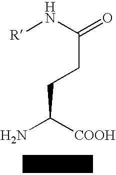 |
| Gln | glutamic acid |
| Gly | glycine |
| His | histidine |
| homoArg | 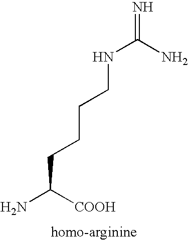 |
| homoCys | 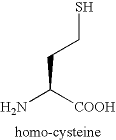 |
| Hyp | 4-hydroxyproline |
| Ile | Isoleucine |
| Leu | Leucine |
| Lys | Lysine |
| Met | Methionine |
| Met(O) | 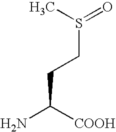 |
| Met(O₂) | 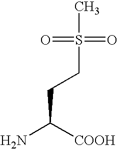 |
| 2-Nal | 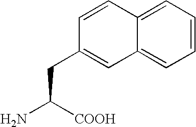 |
| Nle | 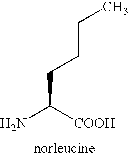 |
| Orn | Ornithine |
| Pen | 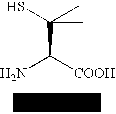 |
| Phe | Phenylalanine |
| Pro | Praline |
| 2-PyAla | 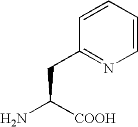 |
| 3-PyAla | 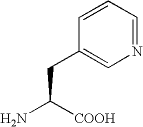 |
| 4-PyAla | 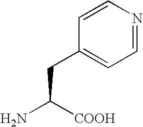 |
| Ser | Serine |
| tBuGly | 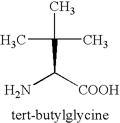 |
| Thr | Threonine |
| (4-thiazolyl)Ala | 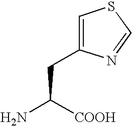 |
| Tic | 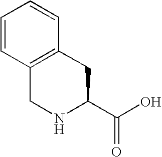 |
| Tyr | Tyrosine |
| Trp | Tryptophan |
| Val | Valine |

Amino acid abbreviations beginning with D- followed by a three letter code, such as D-Ser, D-His and so on, refer to the D-enantiomer of the corresponding amino acid, for example D-serine, D-histidine and so on. In the absence of the letter D preceding a three letter code or amino acid name, as in for example Ser (serine), His (histidine) and so on, it is to be understood that reference is made to the L-enantiomer of the amino acid in question.

### DESCRIPTION OF THE INVENTION

As already indicated above, one aspect of the invention relates to compounds of formula I

R¹—S-Z¹-Z²-Z³-His-Z⁴-Z⁵-c[X¹—X²—X³-Arg-X⁴—X⁵]N(R′)₂ [I]

wherein R¹ represents a straight-chain, branched and/or cyclic C₁₄₋₂₂alkanoyl, C₁₄₋₂₂alkenoyl or C₁₄₋₂₂alkynoyl which may optionally be substituted with one or more substituents selected from halogen, hydroxy and aryl, or R¹ represents C₉₋₁₇—C(O)—NH—S(O)₂—(CH₂)₃—C(O)—;
S represents a bond, a 4-aminobutyric acid residue, Gly, β-Ala or a structure represented by formula II

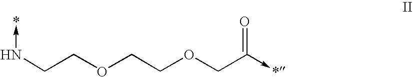

Z¹ represents Gly, β-Ala, Ser, D-Ser, Thr, D-Thr, His, D-His, Asn, D-Asn, Gln, D-Gln, Glu, D-Glu, Asp, D-Asp, Ala or D-Ala;
Z² represents Ser, Thr, Gln, Asn, Glu, Asp or His;
Z³ represents D-Gln or D-Asn;
Z⁴ represents Ser, Thr, Dab, Dap, Glu or Asp;
Z⁵ represents Ala, Val, Leu, Ile, Met or Nle;
X¹ represents Glu, Asp, Cys, homoCys, Pen, Lys, Orn, Dab or Dap;
X² represents His, Cit, Dab, Dap, Cgl, Cha, Val, Ile, tBuGly, Leu, Tyr, Glu, Ala, Nle, Met, Met(O), Met(O₂), Gln, Gln(alkyl), Gln(aryl), Asn, Asn(alkyl), Asn(aryl), Ser, Thr, Cys, Pro, Hyp, Tic, 2-PyAla, 3-PyAla, 4-PyAla, (2-thienyl)alanine, 3-(thienyl)alanine, (4-thiazolyl)Ala, (2-furyl)alanine, (3-furyl)alanine or Phe, wherein the phenyl moiety of said Phe is optionally substituted with a substituent selected among halogen, hydroxy, alkoxy, nitro, benzoyl, methyl, trifluoromethyl, amino and cyano;
X³ represents D-Phe, wherein the phenyl moiety in D-Phe may optionally be substituted with one or more substituents selected among halogen, hydroxy, alkoxy, nitro, methyl, trifluoro-methyl and cyano;
X⁴ represents Trp, 2-Nal, a (3-benzo[b]thienyl)alanine residue or a (S)-2,3,4,9-tetrahydro-1H-β-carboline-3-carboxylic acid residue;
X⁵ represents Glu, Asp, Cys, homoCys, Pen, Lys, Orn, Dab or Dap;
wherein X¹ and X⁵ are joined, rendering the compound of formula I cyclic, either via a disulfide bridge deriving from X¹ and X⁵ both independently being Cys, homoCys or Pen, or via an amide bond formed between a carboxylic acid in the side-chain of X¹ and an amino group in the side chain of X⁵, or between a carboxylic acid in the side-chain of X⁵ and an amino group in the side-chain of X¹;
each R′ independently represents hydrogen or C₁₋₆alkyl, which may optionally be substituted with one or more amino or hydroxy;
with the proviso that the compound of formula I is not tetradecanoyl-Gly-Ser-D-Gln-His-Thr-Nle-c[Glu-Hyp-D-Phe-Arg-Trp-Lys]-NH₂, hexadecanoyl-Gly-Ser-D-Gln-His-Thr-Nle-c[Glu-Hyp-D-Phe-Arg-Trp-Lys]-NH₂, octadecanoyl-Gly-Ser-D-Gln-His-Thr-Nle-c[Glu-Hyp-D-Phe-Arg-Trp-Lys]-NH₂ or hexadecanoyl-Gly-Ser-D-Asn-His-Thr-Nle-c[Glu-Hyp-D-Phe-Arg-Trp-Lys]-NH₂;
and pharmaceutically acceptable salts, prodrugs and solvates thereof.

In certain embodiments of compounds of the invention, R¹ in formula I is C₁₄₋₁₈-alkanoyl, and S is a bond or a structure represented by formula II.

In further embodiments of compounds of the invention, R¹ in formula I is 4-(C₁₄₋₁₈alkanoyl-sulfamoyl)butanoyl, such as 4-(hexadecanoylsulfamoyl)butanoyl, and S is a bond.

In other embodiments of compounds of the invention, Z¹ in formula I is Gly, Glu or Asp, and Z⁵ is Nle or Ala.

In additional embodiments of compounds of the invention, Z² in formula I is Glu, Asp, Ser, Thr, Gln or Asn, notably Ser, Thr, Gln or Asn, in particular Thr or Ser.

In certain embodiments of compounds of the invention, Z³ in formula I is D-Gln. In other embodiments, Z³ is D-Asn.

In still further embodiments of compounds of the invention, Z⁴ in formula I is Glu, Asp, Ser, Thr, Dab or Dap, notably Ser or Thr.

In additional embodiments of compounds of the invention, Z⁵ in formula I is Nle. In other embodiments, Z⁵ is Ala.

In a group of embodiments of compounds of the invention, X¹ in formula I is Glu, X³ in formula I is D-Phe, X⁴ in formula I is Trp, and X⁵ in formula I is Lys. In another group of embodiments, X¹ is Asp, X³ is D-Phe, X⁴ is Trp and X⁵ is Lys.

In certain other embodiments of compounds of the invention, X¹ and X⁵ in formula I are independently Cys, homoCys or Pen, X³ in formula I is D-Phe, and X⁴ in formula I is Trp.

Other relevant embodiments of compounds of the invention include those wherein X² in formula I is Tic, Met(O)₂, Ser, Hyp, Cit, Dap, Asn, Gln or (4-thiazolyl)Ala. Among these, particularly interesting embodiments are embodiments wherein X² is Hyp, embodiments wherein X² is Asn or Gln, and embodiments wherein X² is Cit.

In one particular group of embodiments of compounds of the invention, the moiety N(R′)₂ in formula I is NH₂ (i.e. amino).

Specific types of compounds of the invention of interest include the following:

(SEQ ID NO: 1)

R¹-S-Gly-Ser-D-Gln-His-Ser-Nle-c[Glu-Hyp-D-Phe-
Arg-Trp-Lys]-NH₂,

(SEQ ID NO: 2)

R¹-S-Gly-Thr-D-Gln-His-Ser-Nle-c[Glu-Hyp-D-Phe-
Arg-Trp-Lys]-NH₂,

(SEQ ID NO: 3)

R¹-S-Gly-Gln-D-Gln-His-Ser-Nle-c[Glu-Hyp-D-Phe-
Arg-Trp-Lys]-NH₂,

(SEQ ID NO: 4)

R¹-S-Gly-Ser-D-Asn-His-Ser-Nle-c[Glu-Hyp-D-Phe-
Arg-Trp-Lys]-NH₂,

(SEQ ID NO: 5)

R¹-S-Gly-Thr-D-Asn-His-Ser-Nle-c[Glu-Hyp-D-Phe-
Arg-Trp-Lys]-NH₂,

(SEQ ID NO: 6)

R¹-S-Gly-Gln-D-Asn-His-Ser-Nle-c[Glu-Hyp-D-Phe-
Arg-Trp-Lys]-NH₂,

(SEQ ID NO: 7)

R¹-S-Gly-Ser-D-Gln-His-Thr-Nle-c[Glu-Hyp-D-Phe-
Arg-Trp-Lys]-NH₂,

(SEQ ID NO: 8)

R¹-S-Gly-Thr-D-Gln-His-Thr-Nle-c[Glu-Hyp-D-Phe-
Arg-Trp-Lys]-NH₂,

(SEQ ID NO: 9)

R¹-S-Gly-Gln-D-Gln-His-Thr-Nle-c[Glu-Hyp-D-Phe-
Arg-Trp-Lys]-NH₂,

(SEQ ID NO: 10)

R¹-S-Gly-Ser-D-Asn-His-Thr-Nle-c[Glu-Hyp-D-Phe-
Arg-Trp-Lys]-NH₂,

(SEQ ID NO: 11)

R¹-S-Gly-Thr-D-Asn-His-Thr-Nle-c[Glu-Hyp-D-Phe-
Arg-Trp-Lys]-NH₂,

(SEQ ID NO: 12)

R¹-S-Gly-Gln-D-Asn-His-Thr-Nle-c[Glu-Hyp-D-Phe-
Arg-Trp-Lys]-NH₂,

(SEQ ID NO: 13)

R¹-S-Gly-Ser-D-Gln-His-Ser-Nle-c[Glu-Cit-D-Phe-
Arg-Trp-Lys]-NH₂,

(SEQ ID NO: 14)

R¹-S-Gly-Thr-D-Gln-His-Ser-Nle-c[Glu-Cit-D-Phe-
Arg-Trp-Lys]-NH₂,

(SEQ ID NO: 15)

R¹-S-Gly-Gln-D-Gln-His-Ser-Nle-c[Glu-Cit-D-Phe-
Arg-Trp-Lys]-NH₂,

(SEQ ID NO: 16)

R¹-S-Gly-Ser-D-Asn-His-Ser-Nle-c[Glu-Cit-D-Phe-
Arg-Trp-Lys]-NH₂,

(SEQ ID NO: 17)

R¹-S-Gly-Thr-D-Asn-His-Ser-Nle-c[Glu-Cit-D-Phe-
Arg-Trp-Lys]-NH₂,

(SEQ ID NO: 18)

R¹-S-Gly-Gln-D-Asn-His-Ser-Nle-c[Glu-Cit-D-Phe-
Arg-Trp-Lys]-NH₂,

(SEQ ID NO: 19)

R¹-S-Gly-Ser-D-Gln-His-Thr-Nle-c[Glu-Cit-D-Phe-
Arg-Trp-Lys]-NH₂,

(SEQ ID NO: 20)

R¹-S-Gly-Thr-D-Gln-His-Thr-Nle-c[Glu-Cit-D-Phe-
Arg-Trp-Lys]-NH₂,

(SEQ ID NO: 21)

R¹-S-Gly-Gln-D-Gln-His-Thr-Nle-c[Glu-Cit-D-Phe-
Arg-Trp-Lys]-NH₂,

(SEQ ID NO: 22)

R¹-S-Gly-Ser-D-Asn-His-Thr-Nle-c[Glu-Cit-D-Phe-
Arg-Trp-Lys]-NH₂,

(SEQ ID NO: 23)

R¹-S-Gly-Thr-D-Asn-His-Thr-Nle-c[Glu-Cit-D-Phe-
Arg-Trp-Lys]-NH₂,

(SEQ ID NO: 24)

R¹-S-Gly-Gln-D-Asn-His-Thr-Nle-c[Glu-Cit-D-Phe-
Arg-Trp-Lys]-NH₂,

(SEQ ID NO: 25)

R¹-S-Gly-Ser-D-Gln-His-Ser-Nle-c[Glu-Asn-D-Phe-
Arg-Trp-Lys]-NH₂,

(SEQ ID NO: 26)

R¹-S-Gly-Thr-D-Gln-His-Ser-Nle-c[Glu-Asn-D-Phe-
Arg-Trp-Lys]-NH₂,

(SEQ ID NO: 27)

R¹-S-Gly-Gln-D-Gln-His-Ser-Nle-c[Glu-Asn-D-Phe-
Arg-Trp-Lys]-NH₂,

(SEQ ID NO: 28)

R¹-S-Gly-Ser-D-Asn-His-Ser-Nle-c[Glu-Asn-D-Phe-
Arg-Trp-Lys]-NH₂,

(SEQ ID NO: 29)

R¹-S-Gly-Thr-D-Asn-His-Ser-Nle-c[Glu-Asn-D-Phe-
Arg-Trp-Lys]-NH₂,

(SEQ ID NO: 30)

R¹-S-Gly-Gln-D-Asn-His-Ser-Nle-c[Glu-Asn-D-Phe-
Arg-Trp-Lys]-NH₂,

(SEQ ID NO: 31)

R¹-S-Gly-Ser-D-Gln-His-Thr-Nle-c[Glu-Asn-D-Phe-
Arg-Trp-Lys]-NH₂,

(SEQ ID NO: 32)

R¹-S-Gly-Thr-D-Gln-His-Thr-Nle-c[Glu-Asn-D-Phe-
Arg-Trp-Lys]-NH₂,

(SEQ ID NO: 33)

R¹-S-Gly-Gln-D-Gln-His-Thr-Nle-c[Glu-Asn-D-Phe-
Arg-Trp-Lys]-NH₂,

(SEQ ID NO: 34)

R¹-S-Gly-Ser-D-Asn-His-Thr-Nle-c[Glu-Asn-D-Phe-
Arg-Trp-Lys]-NH₂,

(SEQ ID NO: 35)

R¹-S-Gly-Thr-D-Asn-His-Thr-Nle-c[Glu-Asn-D-Phe-
Arg-Trp-Lys]-NH₂,

(SEQ ID NO: 36)

R¹-S-Gly-Gln-D-Asn-His-Thr-Nle-c[Glu-Asn-D-Phe-
Arg-Trp-Lys]-NH₂,

(SEQ ID NO: 37)

R¹-S-Gly-Ser-D-Gln-His-Ser-Nle-c[homoCys-Hyp-D-
Phe-Arg-Trp-homoCys]-NH₂,

(SEQ ID NO: 38)

R¹-S-Gly-Ser-D-Gln-His-Dap-Nle-c[Cys-Hyp-D-Phe-
Arg-Trp-Pen]-NH₂,

(SEQ ID NO: 39)

R¹-S-Gly-Ser-D-Gln-His-Dap-Nle-c[Cys-Hyp-D-Phe-
Arg-Trp-Cys]-NH₂,

(SEQ ID NO: 40)

R¹-S-Glu-Ser-D-Gln-His-Ser-Nle-c[Glu-Hyp-D-Phe-
Arg-Trp-Lys]-NH₂,

(SEQ ID NO: 41)

R¹-S-Gly-Glu-D-Gln-His-Ser-Nle-c[Glu-Hyp-D-Phe-
Arg-Trp-Lys]-NH₂,

(SEQ ID NO: 42)

R¹-S-Glu-Ser-D-Gln-His-Glu-Nle-c[Glu-Hyp-D-Phe-
Arg-Trp-Lys]-NH₂,
and

(SEQ ID NO: 43)

R¹-S-Gly-Ser-D-Gln-His-Ser-Ala-c[Glu-Hyp-D-Phe-
Arg-Trp-Lys]-NH₂.

wherein R¹ and S may vary as indicated above.

Specific examples of interesting compounds according to the present invention are the following:

(SEQ ID NO: 44)

hexadecanoyl-Gly-Thr-D-Gln-His-Thr-Nle-c[Glu-Hyp-
D-Phe-Arg-Trp-Lys]-NH₂,

(SEQ ID NO: 45)

hexadecanoyl-Gly-Ser-D-Gln-His-Ser-Nle-c[Glu-Hyp-
D-Phe-Arg-Trp-Lys]-NH₂,

(SEQ ID NO: 46)

hexadecanoyl-Gly-Ser-D-Gln-His-Ser-Nle-c[Glu-Dap-
D-Phe-Arg-Trp-Lys]-NH₂,

(SEQ ID NO: 47)

hexadecanoyl-Gly-Ser-D-Gln-His-Ser-Nle-c[Glu-Cit-
D-Phe-Arg-Trp-Lys]-NH₂,

(SEQ ID NO: 48)

hexadecanoyl-Gly-Ser-D-Gln-His-Ser-Nle-c[Glu-Asn-
D-Phe-Arg-Trp-Lys]-NH₂,

(SEQ ID NO: 49)

hexadecanoyl-Gly-Ser-D-Gln-His-Ser-Nle-c[Glu-Gln-
D-Phe-Arg-Trp-Lys]-NH₂,

(SEQ ID NO: 50)

hexadecanoyl-Gly-Gln-D-Gln-His-Ser-Nle-c[Glu-Hyp-
D-Phe-Arg-Trp-Lys]-NH₂,

(SEQ ID NO: 51)

hexadecanoyl-Gly-Gln-D-Asn-His-Ser-Nle-c[Glu-Hyp-
D-Phe-Arg-Trp-Lys]-NH₂,

(SEQ ID NO: 52)

hexadecanoyl-Gly-Ser-D-Asn-His-Ser-Nle-c[Glu-Dap-
D-Phe-Arg-Trp-Lys]-NH₂,

(SEQ ID NO: 53)

hexadecanoyl-Gly-Ser-D-Asn-His-Ser-Nle-c[Glu-Hyp-
D-Phe-Arg-Trp-Lys]-NH₂,

(SEQ ID NO: 54)

octadecanoyl-Gly-Ser-D-Gln-His-Ser-Nle-c[Glu-Dap-
D-Phe-Arg-Trp-Lys]-NH₂,

(SEQ ID NO: 55)

4-(hexadecanoylsulfamoyl)butanoyl-Gly-Ser-D-Gln-
His-Ser-Nle-c[Glu-Hyp-D-Phe-Arg-Trp-Lys]-NH₂,

(SEQ ID NO: 56)

4-(hexadecanoylsulfamoyl)butanoyl-Gly-Ser-D-Asn-
His-Ser-Nle-c[Glu-Hyp-D-Phe-Arg-Trp-Lys]-NH₂,

(SEQ ID NO: 57)

2-[2-(hexadecanoylamino)ethoxy]ethoxyacetyl-Gly-
Thr-D-Gln-His-Ser-Nle-c[Glu-Hyp-D-Phe-Arg-Trp-
Lys]-NH₂,

(SEQ ID NO: 58)

4-(hexadecanoylsulfamoyl)butanoyl-Gly-Thr-D-Gln-
His-Thr-Nle-c[Glu-Hyp-D-Phe-Arg-Trp-Lys]-NH₂,

(SEQ ID NO: 59)

4-(tridecanoylsulfamoyl)butanoyl-Gly-Thr-D-Gln-
His-Thr-Nle-c[Glu-Hyp-D-Phe-Arg-Trp-Lys]-NH₂,

(SEQ ID NO: 60)

hexadecanoyl-Gly-Ser-D-Gln-His-Ser-Nle-c[homoCys-
Hyp-D-Phe-Arg-Trp-homoCys]-NH₂,

(SEQ ID NO: 61)

4-(hexadecanoylsulfamoyl)butanoyl-Gly-Ser-D-Gln-
His-Dap-Nle-c[Cys-Hyp-D-Phe-Arg-Trp-Pen]-NH₂,

(SEQ ID NO: 62)

4-(hexadecanoylsulfamoyl)butanoyl-Gly-Ser-D-Gln-
His-Dap-Nle-c[Cys-Hyp-D-Phe-Arg-Trp-Cys]-NH₂,

(SEQ ID NO: 63)

4-(hexadecanoylsulfamoyl)butanoyl-Glu-Ser-D-Gln-
His-Ser-Nle-c[Glu-Hyp-D-Phe-Arg-Trp-Lys]-NH₂,

(SEQ ID NO: 64)

4-(hexadecanoylsulfamoyl)butanoyl-Gly-Glu-D-Gln-
His-Ser-Nle-c[Glu-Hyp-D-Phe-Arg-Trp-Lys]-NH₂,

(SEQ ID NO: 65)

4-(hexadecanoylsulfamoyl)butanoyl-Glu-Ser-D-Gln-
His-Glu-Nle-c[Glu-Hyp-D-Phe-Arg-Trp-Lys]-NH₂,
and

(SEQ ID NO: 66)

4-(hexadecanoylsulfamoyl)butanoyl-Gly-Ser-D-Gln-
His-Ser-Ala-c[Glu-Hyp-D-Phe-Arg-Trp-Lys]-NH₂.

The present invention also encompasses combinations of two or more embodiments of compounds of the invention as outlined above.

In one aspect of the present invention, the compound of the invention is an agonist of a melanocortin receptor, notably an agonist of MC4. In another aspect of the invention, the compound is a selective agonist of MC4. In this context, selectivity is to be understood in relation to the activity of the compound with respect to MC1, MC3 and/or MC5. If a compound is a significantly more potent as a MC4 agonist than as a MC1, MC3 and/or MC5 agonist, it is deemed to be a selective MC4 agonist. The agonistic potency of a compound with respect to MC1 and MC4 may be determined by comparing an MC1 binding assay as described below under “Assay IV” (MC1) with a functional MC4 assay as described below under “Assay III” (MC4). If a compound is more than 10 times, such as more than 50 times, e.g. more than 100 times more potent with respect to MC4 than with respect to MC1, it is deemed to be a selective MC4 agonist with respect to MC1. The agonistic potency of a compound with respect to MC3, MC4 and MC5 may be determined in functional assays as described in “Assay II” (MC 3 and MC5) and “Assay II” (MC4). If a compound is more than 10 times, such as more than 50 times, e.g. more than 100 times more potent with respect to MC4 than with respect to MC3, it is deemed to be a selective MC4 agonist with respect to MC3. If a compound is more than 10 times, such as more than 50 times, e.g. more than 100 times more potent with respect to MC4 than with respect to MC5, it is deemed to be a selective MC4 agonist with respect to MC5. In a particular aspect, the compound of the present invention is a selective MC4 agonist with respect to MC1, with respect to MC3, with respect to MC5, with respect to MC1 and MC3, with respect to MC1 and MC5, with respect to MC3 and MC5 or with respect to MC1, MC3 and MC5.

In another aspect of the present invention, the compound of the invention is a selective MC4 agonist and a MC3 antagonist. In this context, a compound is deemed to be a selective MC4 agonist and a MC3 antagonist if it is a selective MC4 agonist with respect to MC1 and MC5 as discussed above, and it antagonizes MC3 as determined as described in “Assay II”. In the latter assay, a compound exhibiting an IC₅₀ value of less than 100 nM, such as less than 10 nM, e.g. less than 5 nM, such as less than 1 nM, is deemed to be a MC3 antagonist.

In a further aspect of the present invention, the compound of the present invention is both a selective MC3 agonist and a selective MC4 agonist. In this context, a compound is deemed to be a selective MC3 and MC4 agonist if it is significantly more potent as an agonist towards MC3 and MC4 than as an agonist toward MC1 and MC5. The selectivity of a compound with respect to MC1 and MC3 may be determined by comparing the potency determined for MC1 as described in “Assay IV” with the potency for MC3 determined as described in “Assay II”. If a compound is more than 10 times, such as more than 50 times, e.g. more than 100 times more potent with respect to MC3 than with respect to MC1, it is deemed to be a selective MC3 agonist with respect to MC1. The selectivity of a compound with respect to MC3 and MC5 may be determined by comparing the potency determined as described in “Assay II”. If a compound is more than 10 times, such as more the 50 times, e.g. more than 100 times more potent with respect to MC3 than with respect to MC5, it is deemed to be a selective MC3 agonist with respect to MC5. The MC4 selectivity of a compound with respect to MC3 and MC5 is determined as discussed above.

Compounds of the present invention may exert a protracted effect, i.e. the period of time in which they exert a biological activity may be prolonged. A protracting effect may be evaluated in a slightly modified “Assay I” in a comparison between a compound of the present invention and the corresponding compound wherein R¹ is hydrogen and S is a bond. The experiment is allowed to continue for a period of time, T, until the rats have eaten as much as they did prior to the experiment. T values for compounds of the present invention and the corresponding compounds wherein R¹ is hydrogen and S is a bond are measured, and the difference ΔT is calculated. Compounds of the present invention giving rise to AT above 3 hours, such as above 7 hours, such as above 12 hours, such as above 24 hours, such as above 48 hours, such as above 72 hours, are deemed to exert a protracted effect.

Compounds of the present invention modulate melanocortin receptors, and they are therefore believed to be particularly suited for the treatment of diseases or states which can be treated by a modulation of melanocortin receptor activity. In particular, compounds of the present invention are believed to be suited for the treatment of diseases or states via activation of MC4.

In one aspect, the present invention relates to a method of agonizing or activating MC4 in a subject, the method comprising administering to the subject an effective amount of a compound of the present invention (i.e. a compound of formula I).

In another aspect, the invention provides a method of delaying the progression from impaired glucose tolerance (IGT) to type 2 diabetes, the method comprising administering to a patient in need thereof an effective amount of a compound of the present invention.

In a further aspect, the invention provides a method of delaying the progression from type 2 diabetes to insulin-requiring diabetes, the method comprising administering to a patient in need thereof an effective amount of a compound of the present invention.

In an additional aspect, the invention relates to a method of treating obesity or preventing overweight, the method comprising administering to a patient in need thereof an effective amount of a compound of the present invention.

In a still further aspect, the present invention provides a method of regulating appetite, the method comprising administering to a patient in need thereof an effective amount of a compound of the present invention.

Another aspect of the invention relates to a method of inducing satiety, the method comprising administering to a patient in need thereof an effective amount of a compound of the pre-sent invention.

A further aspect of the invention relates to a method of preventing weight regain after successfully having lost weight, the method comprising administering to a patient in need thereof an effective amount of a compound of the present invention.

Yet another aspect of the invention relates to a method of increasing energy expenditure, the method comprising administering to a patient in need thereof an effective amount of a compound of the present invention.

Still further aspects of the invention include the following:

a method of treating a disease or state related to overweight or obesity, the method comprising administering to a patient in need thereof an effective amount of a compound of the pre-sent invention;
a method of treating bulimia, the method comprising administering to a patient in need thereof an effective amount of a compound of the present invention;
a method of treating binge-eating, the method comprising administering to a patient in need thereof an effective amount of a compound of the present invention;
a method of treating a disease or state selected from atherosclerosis, hypertension, diabetes, type 2 diabetes, impaired glucose tolerance (IGT), dyslipidemia, coronary heart disease, gallbladder disease, gall stone, osteoarthritis, cancer, sexual dysfunction and risk of premature death, the method comprising administering to a patient in need thereof an effective amount of a compound of the present invention.

In particular, compounds of the present invention may be suited for the treatment of diseases in obese or overweight patients. Accordingly, the present invention also provides a method of treating, in an obese patient, a disease or state selected from type 2 diabetes, impaired glucose tolerance (IGT), dyslipidemia, coronary heart disease, gallbladder disease, gall stone, osteoarthritis, cancer, sexual dysfunction and risk of premature death in obese patients, the method comprising administering to an obese patient in need thereof an effective amount of a compound of the present invention.

Moreover, administration of compounds of the present invention may be advantageous in the treatment of patients, notably obese or overweight patients, who have undergone, or are to undergo, gastric banding and/or gastric surgery.

In addition, MC4 agonists could have a positive effect on insulin sensitivity, on drug abuse by modulating the reward system and on hemorrhagic shock. Furthermore, MC3 and MC4 agonists have antipyretic effects, and both have been suggested to be involved in peripheral nerve regeneration. MC4 agonists are also known to reduce stress response.

Appropriate routes of administration of compounds of the invention to patients in the context of the invention include parenteral routes such as nasal, pulmonary or sublingual administration routes, all of which are familiar to persons of skill in the art of drug administration.

In all of the therapeutic methods disclosed above, a compound of the present invention may be administered alone or in combination with one or more (i.e. one or two or three . . . etc.) additional compounds of the present invention. Moreover, a compound of the invention, or a combination of two or more (i.e. two or three or four . . . etc.) compounds of the invention, may be administered in combination with one or more other therapeutically active agents or compounds (i.e. agents or compounds which are not within the scope of the present invention), either sequentially or concomitantly.

A typical dosage of a compound of the invention when employed in a method according to the present invention is in the range of from about 0.001 to about 100 mg/kg body weight per day, e.g. from about 0.01 to about 50 mg/kg body weight per day, such as from about 0.05 to about 10 mg/kg body weight per day, administered in one or more doses, such as from 1 to 3 doses. The exact dosage will depend upon the frequency and mode of administration, the sex, age, weight and general condition of the subject treated, the nature and severity of the condition treated, any concomitant diseases to be treated and other factors evident to those skilled in the art.

Compounds of the invention may conveniently be formulated in unit dosage form using techniques well known to those skilled in the art. A typical unit dosage form intended for oral administration one or more times per day, such as from one to three times per day, may suitably contain from 0.05 to about 1000 mg, e.g. from about 0.1 to about 500 mg, such as from about 0.5 mg to about 200 mg of a compound of the invention.

In a further aspect, the invention relates to a pharmaceutical composition comprising a compound of the present invention, optionally in combination with one or more additional therapeutically active compounds or substances, together with one or more pharmaceutically acceptable carriers or excipients. The composition may suitably be in unit dosage form comprising from about 0.05 mg to about 1000 mg, such as from about 0.1 mg to about 500 mg, e.g. from about 0.5 mg to about 200 mg, of a compound of the present invention.

The present invention also relates to the use of a compound of the present invention, optionally in combination with one or more additional therapeutically active compounds or substances, in the manufacture of a medicament for the treatment of a disease or condition selected from overweight or obesity, bulimia, binge-eating, atherosclerosis, hypertension, type 2 diabetes, impaired glucose tolerance (IGT), dyslipidemia, coronary heart disease, gallbladder disease, gall stone, osteoarthritis, cancer, sexual dysfunction and risk of premature death.

The invention also relates to the use of a compound of the present invention, optionally in combination with one or more additional therapeutically active compounds or substances, in the manufacture of a medicament effective in: delaying the progression from IGT to type 2 diabetes; delaying the progression from type 2 diabetes to insulin-requiring diabetes; regulating appetite; inducing satiety; preventing weight regain after successfully having lost weight; or increasing energy expenditure.

As described above, compounds of the present invention may be administered or applied in combination with one or more additional therapeutically active compounds or substances. Suitable additional compounds or substances may be selected, for example, from antidiabetic agents, antihyperlipidemic agents, antiobesity agents, antihypertensive agents and agents for the treatment of complications resulting from, or associated with, diabetes.

Suitable antidiabetic agents include: insulin; derivatives or analogues of insulin, including derivatives or analogues exhibiting a profile of protracted or prolonged activity, such as those disclosed in WO 95/07931, WO 97/31022 and WO 2005/012347 (Novo Nordisk A/S), the contents of all of which are incorporated herein by reference; derivatives of GLP-1 (glucagon like peptide-1), such as those disclosed in WO 98/08871 (Novo Nordisk A/S), the contents of which are incorporated herein by reference; derivatives of GLP-1 analogues, such as those disclosed in U.S. Pat. No. 6,458,924 (Knudsen et al.), the contents of which are incorporated herein by reference; and orally active hypoglycemic agents.

Suitable orally active hypoglycemic agents include: imidazolines; sulfonylureas; biguanides; meglitinides; oxadiazolidinediones; thiazolidinediones; insulin sensitizers; α-glucosidase inhibitors; agents acting on the ATP-dependent potassium channel of the pancreatic β-cells, e.g. potassium channel openers such as those disclosed in WO 97/26265, WO 99/03861 and WO 00/37474 (Novo Nordisk A/S), the contents of which are incorporated herein by reference; potassium channel openers such as ormitiglinide; potassium channel blockers such as nateglinide or BTS-67582; glucagon antagonists such as those disclosed in WO 99/01423 and WO 00/39088 (Novo Nordisk A/S and Agouron Pharmaceuticals, Inc.), the contents of which are incorporated herein by reference; GLP-1 agonists such as those disclosed in WO 00/42026 (Novo Nordisk A/S and Agouron Pharmaceuticals, Inc.), the contents of which are incorporated herein by reference; DPP-IV (dipeptidyl peptidase-IV) inhibitors; PTPase (protein tyrosine phosphatase) inhibitors; glucokinase activators, such as those described in WO 2004/002481 (Novo Nordisk), the contents of which are incorporated herein by reference, and in WO 02/08209 (Hoffmann La Roche); inhibitors of hepatic enzymes involved in stimulation of gluconeogenesis and/or glycogenolysis; glucose uptake modulators; GSK-3 (glycogen synthase kinase-3) inhibitors; compounds modifying lipid metabolism, such as antihyperlipidemic agents and antilipidemic agents; compounds lowering food intake; as well as PPAR (peroxisome proliferator-activated receptor) agonists and RXR (retinoid X receptor) agonists such as ALRT-268, LG-1268 or LG-1069.

Other examples of suitable additional therapeutically active substances include insulin or insulin analogues; sulfonylureas, e.g. tolbutamide, chlorpropamide, tolazamide, glibenclamide, glipizide, glimepiride, glicazide or glyburide; biguanides, e.g. metformin; and meglitinides, e.g. repaglinide or senaglinide/nateglinide.

Further examples of suitable additional therapeutically active substances include thiazolidinedione insulin sensitizers, e.g. troglitazone, ciglitazone, pioglitazone, rosiglitazone, isaglitazone, darglitazone, englitazone, CS-011/Cl-1037 or T 174, or the compounds disclosed in WO 97/41097 (DRF-2344), WO 97/41119, WO 97/41120, WO 00/41121 and WO 98/45292 (Dr. Reddy's Research Foundation), the contents of all of which are incorporated herein by reference.

Additional examples of suitable additional therapeutically active substances include insulin sensitizers, e.g. GI 262570, YM-440, MCC-555, JTT-501, AR-H039242, KRP-297, GW-409544, CRE-16336, AR-H049020, LY510929, MBX-102, CLX-0940, GW-501516 and the compounds disclosed in WO 99/19313 (NN622/DRF-2725), WO 00/50414, WO 00/63191, WO 00/63192 and WO 00/63193 (Dr. Reddy's Research Foundation), and in WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), the contents of all of which are incorporated herein by reference.

Still further examples of suitable additional therapeutically active substances include:

α-glucosidase inhibitors, e.g. voglibose, emiglitate, miglitol or acarbose;
glycogen phosphorylase inhibitors, e.g. the compounds described in WO 97/09040 (Novo Nordisk A/S);
glucokinase activators;
agents acting on the ATP-dependent potassium channel of the pancreatic β-cells, e.g. tolbutamide, glibenclamide, glipizide, glicazide, BTS-67582 or repaglinide;

Other suitable additional therapeutically active substances include antihyperlipidemic agents and antilipidemic agents, e.g. cholestyramine, colestipol, clofibrate, gemfibrozil, lovastatin, pravastatin, simvastatin, probucol or dextrothyroxine.

Further agents which are suitable as additional therapeutically active substances include antiobesity agents and appetite-regulating agents. Such substances may be selected from the group consisting of CART (cocaine amphetamine regulated transcript) agonists, NPY (neuropeptide Y) antagonists, Y2 and Y4 receptor agonists, MC3 (melanocortin 3) agonists, MC3 (melanocortin 3) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, α3 adrenergic agonists such as CL-316243, AJ-9677, GW-0604, LY362884, LY377267 or AZ-40140, MC1 (melanocortin 1) agonists, MCH (melanocyte-concentrating hormone) antagonists, CCK (cholecystokinin) agonists, serotonin reuptake inhibitors (e.g. fluoxetine, seroxat or citalopram), serotonin and norepinephrine reuptake inhibitors, 5HT (serotonin) agonists, bombesin agonists, galanin antagonists, growth hormone, growth factors such as prolactin or placental lactogen, growth hormone releasing compounds (growth hormone secretagogues), ghrelin antagonists, TRH (thyrotropin releasing hormone) agonists, UCP 2 or 3 (uncoupling protein 2 or 3) modulators, chemical uncouplers, leptin agonists, DA (dopamine) agonists (bromocriptin, doprexin), lipase/amylase inhibitors, PPAR modulators, RXR modulators, TR β agonists, adrenergic CNS stimulating agents, AGRP (agouti-related protein) inhibitors, histamine H3 receptor antagonists such as those disclosed in WO 00/42023, WO 00/63208 and WO 00/64884, the contents of all of which are incorporated herein by reference, exendin-4, GLP-1 agonists and ciliary neurotrophic factor.

Further suitable antiobesity agents are bupropion (antidepressant), topiramate (anticonvulsant), ecopipam (dopamine D1/D5 antagonist), naltrexone (opioid antagonist), and peptide YY3-36 (Batterham et al, Nature 418, 650-654 (2002)).

An embodiment of a suitable antiobesity agent for use in a method of the invention as an additional therapeutically active substance in combination with a compound of the invention is leptin.

A further embodiment of a suitable antiobesity agent is peptide YY3-36. Additional embodiments of suitable antiobesity agents are serotonin and norepinephrine reuptake inhibitors, e.g. sibutramine.

Other embodiments of suitable antiobesity agents are lipase inhibitors, e.g. orlistat.

Still further embodiments of suitable antiobesity agents are adrenergic CNS stimulating agents, e.g. dexamphetamine, amphetamine, phentermine, mazindol, phendimetrazine, diethylpropion, fenfluramine or dexfenfluramine.

Other examples of suitable additional therapeutically active compounds include antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin.

In certain embodiments of the uses and methods of the present invention, the compound of the present invention may be administered or applied in combination with more than one of the above-mentioned, suitable additional therapeutically active compounds or substances, e.g. in combination with: metformin and a sulfonylurea such as glyburide; a sulfonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

### Pharmaceutical Compositions

As already mentioned, one aspect of the present invention provides a pharmaceutical composition (formulation) comprising a compound of the present invention. Appropriate embodiments of such formulations will often contain a compound of the invention in a concentration of from 10⁻³ mg/ml to 200 mg/ml, such as, e.g., from 10⁻¹ mg/ml to 100 mg/ml. The pH in such a formulation of the invention will typically be in the range of 2.0 to 10.0. The formulation may further comprise a buffer system, preservative(s), tonicity agent(s), chelating agent(s), stabilizer(s) and/or surfactant(s). In one embodiment of the invention the pharmaceutical formulation is an aqueous formulation, i.e. a formulation comprising water, and the term “aqueous formulation” in the present context may normally be taken to indicate a formulation comprising at least 50% by weight (w/w) of water. Such a formulation is typically a solution or a suspension. An aqueous formulation of the invention in the form of an aqueous solution will normally comprise at least 50% (w/w) of water. Likewise, an aqueous formulation of the invention in the form of an aqueous suspension will normally comprise at least 50% (w/w) of water.

In another embodiment, a pharmaceutical composition (formulation) of the invention may be a freeze-dried (i.e. lyophilized) formulation intended for reconstitution by the physician or the patient via addition of solvents and/or diluents prior to use.

In a further embodiment, a pharmaceutical composition (formulation) of the invention may be a dried formulation (e.g. freeze-dried or spray-dried) ready for use without any prior dissolution.

In a further aspect, the invention relates to a pharmaceutical composition (formulation) comprising an aqueous solution of a compound of the present invention, and a buffer, wherein the compound of the invention is present in a concentration of 0.1-100 mg/ml or above, and wherein the formulation has a pH from about 2.0 to about 10.0.

In another embodiment of the invention, the pH of the formulation has a value selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 and 10.0.

In a further embodiment, the buffer in a buffered pharmaceutical composition of the invention may comprise one or more buffer substances selected from the group consisting of sodium acetate, sodium carbonate, citrates, glycylglycine, histidine, glycine, lysine, arginine, sodium dihydrogen phosphate, disodium hydrogen phosphate, sodium phosphate, tris(hydroxymethyl)aminomethane (TRIS), bicine, tricine, malic acid, succinates, maleic acid, fumaric acid, tartaric acid and aspartic acid. Each one of these specific buffers constitutes an alternative embodiment of the invention.

In another embodiment, a pharmaceutical composition of the invention may comprise a pharmaceutically acceptable preservative, e.g. one or more preservatives selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, thiomerosal, bronopol, benzoic acid, imidurea, chlorohexidine, sodium dehydroacetate, chlorocresol, ethyl p-hydroxybenzoate, benzethonium chloride and chlorphenesine (3p-chlorphenoxypropane-1,2-diol). Each one of these specific preservatives constitutes an alternative embodiment of the invention. In a further embodiment of the invention the preservative is present in a concentration from 0.1 mg/ml to 20 mg/ml. In still further embodiments of such a pharmaceutical composition of the invention, the preservative is present in a concentration in the range of 0.1 mg/ml to 5 mg/ml, a concentration in the range of 5 mg/ml to 10 mg/ml, or a concentration in the range of 10 mg/ml to 20 mg/ml. The use of a preservative in pharmaceutical compositions is well known to the skilled person. For convenience, reference is made in this respect to Remington: The Science and Practice of Pharmacy, 20^{th} edition, 2000.

In a further embodiment of the invention the formulation further comprises a tonicity-adjusting agent, i.e. a substance added for the purpose of adjusting the tonicity (osmotic pressure) of a liquid formulation (notably an aqueous formulation) or a reconstituted freeze-dried formulation of the invention to a desired level, normally such that the resulting, final liquid formulation is isotonic or substantially isotonic. Suitable tonicity-adjusting agents may be selected from the group consisting of salts (e.g. sodium chloride), sugars and sugar alcohols (e.g. mannitol), amino acids (e.g. glycine, histidine, arginine, lysine, isoleucine, aspartic acid, tryptophan or threonine), alditols [e.g. glycerol (glycerine), 1,2-propanediol (propyleneglycol), 1,3-propanediol or 1,3-butanediol], polyethyleneglycols (e.g. PEG 400) and mixtures thereof.

Any sugar, such as a mono-, di- or polysaccharide, or a water-soluble glucan, including for example fructose, glucose, mannose, sorbose, xylose, maltose, lactose, sucrose, trehalose, dextran, pullulan, dextrin, cyclodextrin, soluble starch, hydroxyethyl starch or carboxymethylcellulose-sodium, may be used; in one embodiment, sucrose may be employed. Sugar alcohols (polyols derived from mono-, di-, oligo- or polysaccharides) include, for example, mannitol, sorbitol, inositol, galactitol, dulcitol, xylitol, and arabitol. In one embodiment, the sugar alcohol employed is mannitol. Sugars or sugar alcohols mentioned above may be used individually or in combination. There is no fixed limit to the amount used, as long as the sugar or sugar alcohol is soluble in the liquid composition (formulation) and does not adversely effect the stabilizing effects achieved using the methods of the invention. In one embodiment, the concentration of sugar or sugar alcohol is between about 1 mg/ml and about 150 mg/ml.

In further embodiments, the tonicity-adjusting agent is present in a concentration of from 1 mg/ml to 50 mg/ml, such as from 1 mg/ml to 7 mg/ml, from 8 mg/ml to 24 mg/ml, or from 25 mg/ml to 50 mg/ml. A pharmaceutical composition of the invention containing any of the tonicity-adjusting agents specifically mentioned above constitutes an embodiment of the invention. The use of a tonicity-adjusting agent in pharmaceutical compositions is well known to the skilled person. For convenience, reference is made to Remington: The Science and Practice of Pharmacy, 20^{th} edition, 2000.

In a still further embodiment of a pharmaceutical composition (formulation) of the invention, the formulation further comprises a chelating agent. Suitable chelating agents may be selected, for example, from salts of ethylenediaminetetraacetic acid (EDTA), citric acid, and aspartic acid, and mixtures thereof. The concentration of chelating agent will suitably be in the range from 0.1 mg/ml to 5 mg/ml, such as from 0.1 mg/ml to 2 mg/ml or from 2 mg/ml to 5 mg/ml. A pharmaceutical composition of the invention containing any of the chelating agents specifically mentioned above constitutes an embodiment of the invention. The use of a chelating agent in pharmaceutical compositions is well known to the skilled person. For convenience, reference is made to Remington: The Science and Practice of Pharmacy, 20^{th} edition, 2000.

In another embodiment of a pharmaceutical composition (formulation) of the invention, the formulation further comprises a stabilizer. The use of a stabilizer in pharmaceutical compositions is well known to the skilled person. For convenience, reference is made to Remington: The Science and Practice of Pharmacy, 20^{th} edition, 2000.

More particularly, particularly useful compositions of the invention include stabilized liquid pharmaceutical compositions whose therapeutically active components include an oligo- or polypeptide that possibly exhibits aggregate formation during storage in liquid pharmaceutical formulations. By “aggregate formation” is meant the formation of oligomers, which may remain soluble, or large visible aggregates that precipitate from the solution, as the result of a physical interaction between the oligo- or polypeptide molecules. The term “during storage” refers to the fact that a liquid pharmaceutical composition or formulation, once prepared, is not normally administered to a subject immediately. Rather, following preparation, it is packaged for storage, whether in a liquid form, in a frozen state, or in a dried form for later reconstitution into a liquid form or other form suitable for administration to a subject. By “dried form” is meant the product obtained when a liquid pharmaceutical composition or formulation is dried by freeze-drying (i.e., lyophilization; see, for example, Williams and Polli (1984) J. Parenteral Sci. Technol. 38: 48-59), by spray-drying [see, e.g., Masters (1991) in Spray-Drying Handbook (5th edn.; Longman Scientific and Technical, Essex, U.K.), pp. 491-676; Broadhead et al. (1992) Drug Devel. Ind. Pharm. 18: 1169-1206; and Mumenthaler et al. (1994) Pharm. Res. 11: 12-20], or by air-drying [see, e.g., Carpenter and Crowe (1988) Cryobiology 25: 459-470; and Roser (1991) Biopharm. 4: 47-53]. Aggregate formation by an oligo- or polypeptide during storage of a liquid pharmaceutical composition can adversely affect biological activity of that peptide, resulting in loss of therapeutic efficacy of the pharmaceutical composition. Furthermore, aggregate formation may cause other problems, such as blockage of tubing, membranes or pumps when the oligo- or polypeptide-containing pharmaceutical composition is administered using an infusion system.

A pharmaceutical composition of the invention may further comprise an amount of an amino acid base sufficient to decrease aggregate formation by the oligo- or polypeptide during storage of the composition. By “amino acid base” is meant an amino acid, or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids for use in preparing a composition of the invention are those carrying a charged side chain, such as arginine, lysine, aspartic acid and glutamic acid. Any stereoisomer (i.e., L, D, or mixtures thereof) of a particular amino acid (e.g. methionine, histidine, arginine, lysine, isoleucine, aspartic acid, tryptophan or threonine, and mixtures thereof) or combinations of these stereoisomers, may be present in the pharmaceutical compositions of the invention so long as the particular amino acid is present either in its free base form or its salt form. In one embodiment, the L-stereoisomer of an amino acid is used. Compositions of the invention may also be formulated with analogues of these amino acids. By “amino acid analogue” is meant a derivative of a naturally occurring amino acid that brings about the desired effect of decreasing aggregate formation by the oligo- or polypeptide during storage of liquid pharmaceutical compositions of the invention. Suitable arginine analogues include, for example, aminoguanidine, ornithine and N-monoethyl-L-arginine. Suitable methionine analogues include ethionine and buthionine, and suitable cysteine analogues include S-methyl-L-cysteine. As with the amino acids per se, amino acid analogues are incorporated into compositions of the invention in either their free base form or their salt form. In a further embodiment of the invention, the amino acids or amino acid analogues are incorporated in a concentration which is sufficient to prevent or delay aggregation of the oligo- or polypeptide.

In a particular embodiment of the invention, methionine (or another sulfur-containing amino acid or amino acid analogue) may be incorporated in a composition of the invention to inhibit oxidation of methionine residues to methionine sulfoxide when the oligo- or polypeptide acting as the therapeutic agent is a peptide comprising at least one methionine residue susceptible to such oxidation. The term “inhibit” in this context refers to minimization of accumulation of methionine-oxidized species over time. Inhibition of methionine oxidation results in increased retention of the oligo- or polypeptide in its proper molecular form. Any stereoisomer of methionine (L or D) or combinations thereof can be used. The amount to be added should be an amount sufficient to inhibit oxidation of methionine residues such that the amount of methionine sulfoxide is acceptable to regulatory agencies. Typically, this means that no more than from about 10% to about 30% of forms of the oligo- or polypeptide wherein methionine is sulfoxidated are present. In general, this can be achieved by incorporating methionine in the composition such that the ratio of added methionine to methionine residues ranges from about 1:1 to about 1000:1, such as from about 10:1 to about 100:1.

In a further embodiment of the invention the formulation further comprises a stabilizer selected from high-molecular-weight polymers and low-molecular-weight compounds. Thus, for example, the stabilizer may be selected from substances such as polyethylene glycol (e.g. PEG 3350), polyvinyl alcohol (PVA), polyvinylpyrrolidone, carboxy-/hydroxycellulose and derivatives thereof (e.g. HPC, HPC-SL, HPC-L or HPMC), cyclodextrins, sulfurcontaining substances such as monothioglycerol, thioglycolic acid and 2-methylthioethanol, and various salts (e.g. sodium chloride). A pharmaceutical composition of the invention containing any of the stabilizers specifically mentioned above constitutes an embodiment of the invention.

Pharmaceutical compositions of the present invention may also comprise additional stabilizing agents which further enhance stability of a therapeutically active oligo- or polypeptide therein. Stabilizing agents of particular interest in the context of the present invention include, but are not limited to: methionine and EDTA, which protect the peptide against methionine oxidation; and surfactants, notably nonionic surfactants, which protect the polypeptide against aggregation or degradation associated with freeze-thawing or mechanical shearing.

Thus, in a further embodiment of the invention, the pharmaceutical formulation comprises a surfactant, particularly a nonionic surfactant. Examples thereof include ethoxylated castor oil, polyglycolyzed glycerides, acetylated monoglycerides, sorbitan fatty acid esters, polyoxypropylene-polyoxyethylene block polymers (e.g. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (Tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (e.g. phosphatidyl-serine, phosphatidyl-choline, phosphatidyl-ethanolamine, phosphatidyl-inositol, diphosphatidyl-glycerol and sphingomyelin), derivatives of phospholipids (e.g. dipalmitoyl phosphatidic acid) and lysophospholipids (e.g. palmitoyl lysophosphatidyl-L-serine and 1-acyl-sn-glycero-3-phosphate esters of ethanolamine, choline, serine or threonine) and alkyl, alkyl ester and alkyl ether derivatives of lysophosphatidyl and phosphatidylcholines, e.g. lauroyl and myristoyl derivatives of lysophosphatidylcholine, dipalmitoylphosphatidylcholine, and modifications of the polar head group, i.e. cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (e.g. galactopyranoside), sphingoglycolipids (e.g. ceramides, gangliosides), dodecylphosphocholine, hen egg lysolecithin, fusidic acid derivatives (e.g. sodium tauro-dihydrofusidate, etc.), long-chain fatty acids (e.g. oleic acid or caprylic acid) and salts thereof, acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no. [577-11-7]), docusate calcium, CAS registry no. [128-49-4]), docusate potassium, CAS registry no. [7491-09-0]), SDS (sodium dodecyl sulfate or sodium lauryl sulfate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-hexadecyl-N,N-dimethyl-3-ammonio-1-propanesulfonate, anionic (alkyl-aryl-sulfonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (e.g. Tetronic's), which are tetrafunctional block copolymers derived from sequential addition of propylene oxide and ethylene oxide to ethylenediamine. The surfactant may also be selected from imidazoline derivatives and mixtures thereof. A pharmaceutical composition of the invention containing any of the surfactants specifically mentioned above constitutes an embodiment of the invention.

The use of a surfactant in pharmaceutical compositions is well-known to the skilled person. For convenience, reference is made to Remington: The Science and Practice of Pharmacy, 20^{th} edition, 2000.

Additional ingredients may also be present in a pharmaceutical composition (formulation) of the present invention. Such additional ingredients may include, for example, wetting agents, emulsifiers, antioxidants, bulking agents, metal ions, oleaginous vehicles, proteins (e.g. human serum albumin, gelatine or other proteins) and a zwitterionic species (e.g. an amino acid such as betaine, taurine, arginine, glycine, lysine or histidine). Such additional ingredients should, of course, not adversely affect the overall stability of the pharmaceutical formulation of the present invention.

Pharmaceutical compositions containing a compound according to the present invention may be administered to a patient in need of such treatment at one or more of several sites, for example at topical sites (e.g. skin and mucosal sites), at sites which bypass absorption (e.g. via administration in an artery, in a vein or in the heart), and at sites which involve absorption (e.g. in the skin, under the skin, in a muscle or in the abdomen).

Administration of pharmaceutical compositions according to the invention to patients in need thereof may be via several routes of administration. These include, for example, lingual, sublingual, buccal, in the mouth, oral, in the stomach and intestine, nasal, pulmonary (for example through the bronchioles and alveoli or a combination thereof), epidermal, dermal, transdermal, vaginal, rectal, ocular (for example through the conjunctiva), urethral and parenteral.

Compositions of the present invention may be administered in various dosage forms, for example in the form of solutions, suspensions, emulsions, microemulsions, multiple emulsion, foams, salves, pastes, plasters, ointments, tablets, coated tablets, rinses, capsules (e.g. hard gelatine capsules or soft gelatine capsules), suppositories, rectal capsules, drops, gels, sprays, powder, aerosols, inhalants, eye drops, ophthalmic ointments, ophthalmic rinses, vaginal pessaries, vaginal rings, vaginal ointments, injection solutions, in situ-transforming solutions (for example in situ gelling, in situ setting, in situ precipitating or in situ crystallizing), infusion solutions or implants.

Compositions of the invention may further be compounded in, or bound to, e,g. via covalent, hydrophobic or electrostatic interactions, a drug carrier, drug delivery system or advanced drug delivery system in order to further enhance the stability of the compound of the present invention, increase bioavailability, increase solubility, decrease adverse effects, achieve chronotherapy well known to those skilled in the art, and increase patient compliance, or any combination thereof. Examples of carriers, drug delivery systems and advanced drug delivery systems include, but are not limited to: polymers, for example cellulose and derivatives; polysaccharides, for example dextran and derivatives, starch and derivatives; poly(vinyl alcohol); acrylate and methacrylate polymers; polylactic and polyglycolic acid and block copolymers thereof; polyethylene glycols; carrier proteins, for example albumin; gels, for example thermogelling systems, such as block co-polymeric systems well known to those skilled in the art; micelles; liposomes; microspheres; nanoparticulates; liquid crystals and dispersions thereof; L2 phase and dispersions thereof well known to those skilled in the art of phase behaviour in lipid-water systems; polymeric micelles; multiple emulsions (self-emulsifying, self-microemulsifying); cyclodextrins and derivatives thereof; and dendrimers.

Compositions of the present invention are useful in the formulation of solids, semisolids, powders and solutions for pulmonary administration of a compound of the present invention, using, for example, a metered dose inhaler, dry powder inhaler or a nebulizer, all of which are devices well known to those skilled in the art.

Compositions of the present invention are useful in the formulation of controlled-release, sustained-release, protracted, retarded or slow-release drug delivery systems. Compositions of the invention are thus of value in the formulation of parenteral controlled-release and sustained-release systems well known to those skilled in the art (both types of systems leading to a many-fold reduction in the number of administrations required).

Of particular value are controlled-release and sustained-release systems for subcutaneous administration. Without limiting the scope of the invention, examples of useful controlled release systems and compositions are those containing hydrogels, oleaginous gels, liquid crystals, polymeric micelles, microspheres or nanoparticles,

Methods for producing controlled-release systems useful for compositions of the present invention include, but are not limited to, crystallization, condensation, co-crystallization, precipitation, co-precipitation, emulsification, dispersion, high-pressure homogenisation, encapsulation, spray-drying, microencapsulation, coacervation, phase separation, solvent evaporation to produce microspheres, extrusion and supercritical fluid processes. General reference is made in this context to Handbook of Pharmaceutical Controlled Release (Wise, D. L., ed. Marcel Dekker, New York, 2000), and Drugs and the Pharmaceutical Sciences, vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, for example a syringe in the form of a pen device. Alternatively, parenteral administration may be performed by means of an infusion pump. A further option is administration of a composition of the invention which is a liquid (typically aqueous) solution or suspension in the form of a nasal or pulmonary spray. As a still further option, a pharmaceutical composition of the invention can be adapted to transdermal administration (e.g. by needle-free injection or via a patch, such as an iontophoretic patch) or transmucosal (e.g. buccal) administration.

The term “stabilized formulation” refers to a formulation with increased physical stability, increased chemical stability or increased physical and chemical stability. The term “physical stability” in the context of a formulation containing an oligo- or polypeptide refers to the tendency of the peptide to form biologically inactive and/or insoluble aggregates as a result of exposure to thermo-mechanical stresses and/or interaction with interfaces and surfaces that are destabilizing, such as hydrophobic surfaces and interfaces. Physical stability of aqueous protein formulations is evaluated by means of visual inspection and/or turbidity measurements after exposing the formulation, filled in suitable containers (e.g. cartridges or vials), to mechanical/physical stress (e.g. agitation) at different temperatures for various time periods. Visual inspection of formulations is performed in a sharp focused light with a dark background. The turbidity of a formulation is characterized by a visual score ranking the degree of turbidity, for instance on a scale from 0 to 3 (in that a formulation showing no turbidity corresponds to a visual score 0, whilst a formulation showing visual turbidity in daylight corresponds to visual score 3). A formulation is normally classified physically unstable with respect to aggregation when it shows visual turbidity in daylight. Alternatively, the turbidity of a formulation can be evaluated by simple turbidity measurements well-known to the skilled person. Physical stability of aqueous oligo- or polypeptide formulations can also be evaluated by using a spectroscopic agent or probe of the conformational status of the peptide. The probe is preferably a small molecule that preferentially binds to a non-native conformer of the oligo- or polypeptide. One example of a small-molecular spectroscopic probe of this type is Thioflavin T. Thioflavin T is a fluorescent dye that has been widely used for the detection of amyloid fibrils. In the presence of fibrils, and possibly also other configurations, Thioflavin T gives rise to a new excitation maximum at about 450 nm, and enhanced emission at about 482 nm when bound to a fibril form. Unbound Thioflavin T is essentially non-fluorescent at the wavelengths in question.

Other small molecules can be used as probes of the changes in peptide structure from native to non-native states. Examples are the “hydrophobic patch” probes that bind preferentially to exposed hydrophobic patches of a polypeptide. The hydrophobic patches are generally buried within the tertiary structure of a polypeptide in its native state, but become exposed as it begins to unfold or denature. Examples of such small-molecular, spectroscopic probes are aromatic, hydrophobic dyes, such as anthracene, acridine, phenanthroline and the like. Other spectroscopic probes are metal complexes of amino acids, such as cobalt complexes of hydrophobic amino acids, e.g. phenylalanine, leucine, isoleucine, methionine, valine, or the like.

The term “chemical stability” of a pharmaceutical formulation as used herein refers to chemical covalent changes in oligo- or polypeptide structure leading to formation of chemical degradation products with potentially lower biological potency and/or potentially increased immunogenicity compared to the original molecule. Various chemical degradation products can be formed depending on the type and nature of the starting molecule and the environment to which it is exposed. Elimination of chemical degradation can most probably not be completely avoided and gradually increasing amounts of chemical degradation products may often be seen during storage and use of oligo- or polypeptide formulations, as is well known to the person skilled in the art. A commonly encountered degradation process is deamidation, a process in which the side-chain amide group in glutaminyl or asparaginyl residues is hydrolysed to form a free carboxylic acid. Other degradation pathways involve formation of higher molecular weight transformation products wherein two or more molecules of the starting sub-stance are covalently bound to each other through transamidation and/or disulfide interactions, leading to formation of covalently bound dimer, oligomer or polymer degradation products (see, e.g., Stability of Protein Pharmaceuticals, Ahern. T. J. & Manning M. C., Plenum Press, New York 1992). Oxidation (of for instance methionine residues) may be mentioned as another variant of chemical degradation. The chemical stability of a formulation may be evaluated by measuring the amounts of chemical degradation products at various time-points after exposure to different environmental conditions (in that the formation of degradation products can often be accelerated by, e.g., increasing temperature). The amount of each individual degradation product is often determined by separation of the degradation products depending on molecule size and/or charge using various chromatographic techniques (e.g. SEC-HPLC and/or RP-HPLC).

Hence, as outlined above, a “stabilized formulation” refers to a formulation with increased physical stability, increased chemical stability, or increased physical and chemical stability. In general, a pharmaceutical composition (formulation) must be stable during use and storage (in compliance with recommended use and storage conditions) until the expiry date is reached.

A pharmaceutical composition (formulation) of the invention should preferably be stable for more than 2 weeks of usage and for more than two years of storage, more preferably for more than 4 weeks of usage and for more than two years of storage, desirably for more than 4 weeks of usage and for more than 3 years of storage, and most preferably for more than 6 weeks of usage and for more than 3 years of storage.

All references, including publications, patent applications and patents, cited herein are hereby incorporated by reference in their entirety and to the same extent as if each reference were individually and specifically indicated to be incorporated by reference and were set forth in its entirety herein (to the maximum extent permitted by law).

Headings and sub-headings are used herein for convenience only, and should not be construed as limiting the invention in any way.

The use of any and all examples, or exemplary language (including “for instance”, “for example”, “e.g.” and “such as”) in the present specification is intended merely to better illuminate the invention, and does not pose a limitation on the scope of the invention unless otherwise indicated. No language in the specification should be construed as indicating any non-claimed element as being essential to the practice of the invention.

The citation and incorporation of patent documents herein is done for convenience only, and does not reflect any view of the validity, patentability and/or enforceability of such patent documents.

The present invention includes all modifications and equivalents of the subject matter recited in the claims appended hereto, as permitted by applicable law.

### EXAMPLES

### List of Abbreviations Employed

AcOH acetic acid
BSA bovine serum albumin
DCM dichloromethane
DIC diisopropylcarbodiimide
DIPEA ethyldiisopropylamine

### DMAP 4-N,N-dimethylaminopyridine

### DMEM Dulbecco's Modified Eagle's Medium

### DMF N,N-dimethylformamide

DMSO dimethyl sulfoxide
EGTA 1,2-di(2-aminoethoxy)ethane-N,N,N′,N′-tetraacetic acid
FCS fetal calf serum
Fmoc 9-fluorenylmethyloxycarbonyl
HEPES 2-[4-(2-hydroxyethyl)-piperazin-1-yl]-ethanesulfonic acid
HOAt 1-hydroxy-7-azabenzotriazole
HOBt 1-hydroxybenzotriazole
HSA human serum albumin
MeCN acetonitrile
MeOH methanol
α-MSH α-form of melanocyte-stimulating hormone
MTX methotrexate
NEt₃ triethylamine

### NMP N-methylpyrrolidone

PBS phosphate-buffered saline
PEI polyethyleneimine
PhMe toluene
PyBop (benzotriazol-1-yloxy)tripyrrolidino-phosphonium hexafluorophosphate

All compounds of the present invention can be synthesized by those skilled in the art using standard coupling and deprotection steps. A description of all necessary tools and synthetic methods including standard abbreviations for peptide synthesis can be found in “The Fine Art Of Solid Phase Synthesis”, 2002/3 Catalogue, Novabiochem.

In the examples listed below, Rt values are retention times and the mass values are those detected by the mass spectroscopy (MS) detector and obtained using the following HPLC-MS device (LCMS):

### LCMS

Sciex API-150 Ex Quadrupole MS, electrospray, m/z=200 to m/z=1500; column: Waters XTerra® MS C₁₈ 5 μm 3.0×50 mm; elution with a mixture of solution A (water containing 0.1% TFA) and solution B (acetonitrile containing 0.08% TFA); gradient: 5%→20% solution B from 1.0 to 3.0 min, 20%→50% solution B from 3.0 to 16.0 min, 50%→90% solution B from 16.0 to 18.0 min, elution until t=18.0 min; flow 1.5 ml/min. UV detection at 214 nm.

Calculation of the peptide concentration was based on the absorbance of the Trp residue at 280 nm in a 1-cm cell:

(milligrams of peptide) per milliliter=(absorbance×dilution factor×molecular weight)/(number of Trp residues×5560 ml/mmol).

### Example 1

### Hexadecanoyl-Gly-Thr-D-Gln-His-Thr-Nle-c[Glu-Hyp-D-Phe-Arg-Trp-Lys]-NH₂

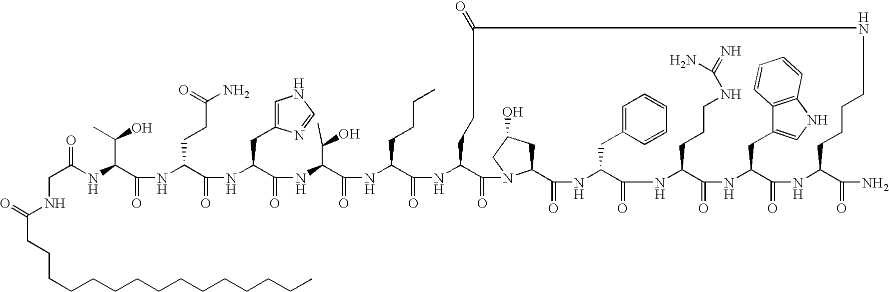

The protected peptidyl resin Fmoc-Nle-Glu(2-phenylisopropyloxy)-Hyp(tBu)-D-Phe-Arg(Pbf)-Trp(Boc)-Lys(Mtt)-NH-Rink linker polystyrene was synthesized from 3.25 mmol of commercially available Rink amide polystyrene resin. The synthesis was performed according to the Fmoc strategy on a MultiSyntech semi-automatic synthesizer equipped with one reactor. A protocol which employs (1) 0.45 M HBTU in DMF, (2) 2 M DIPEA in NMP and (3) a 0.5 M solution of the protected Fmoc-amino acid solvated in a 0.5 M solution of HOBt/HOAt (1:1) in NMP was employed. The amino acids were pre-activated by mixing the three solutions [18 ml (1), 14 ml (2) and 18 ml (3)] and agitating the mixture for 10 min. Reactions were run for 2 h with the exception of the attachment of the first amino acid Fmoc-Lys(Mtt)-OH, which was repeated once. The resin was washed 3 times with 60 ml NMP before removal of the Fmoc protection group. Fmoc-removal was performed by treating the resin with 50 ml of 20% piperidine in NMP twice for 5 and 15 min., respectively, followed by 6×60 ml NMP wash. The Fmoc-removal and coupling steps were repeated until the desired sequence was obtained.

For deprotection of the side chains of Glu and Lys, the peptidyl resin was washed with 3×60 ml DCM and then treated for 6×10 min with 50 ml of 2% trifluoroacetic acid (TFA), 2.5% TES in DCM, with regular mixing. The resin was washed with 5×50 ml DCM, NMP with 5% DIPEA and NMP. The peptide was cyclized using HOBt (13 mmol), PyBOP (13 mmol) and DIPEA (39 mmol) in DMF (50 ml) with regular mixing for 16 h. The resin was washed with 4×60 ml NMP and 6×60 ml DCM.

The remaining five carboxylic acids Fmoc-Thr(tBu)-OH, Fmoc-His(Trt)-OH, Fmoc-D-Gln(Trt)-OH, Fmoc-Thr(tBu)-OH, Fmoc-Gly-OH and hexadecanoic acid (palmitic acid) were added according to the procedure described above.

The peptide was cleaved from the resin by stirring for 2 hours at room temperature with 35 ml TFA with 2.5% water and 2.5% TES. The cleavage mixture was filtered, and the resin was washed with an additional 10 ml of TFA. The crude peptide was precipitated from the combined filtrate by addition of 400 ml diethyl ether and isolated by centrifugation. The precipitate was washed twice with diethyl ether.

The crude cyclic peptide was purified by preparative RP-HPLC in accordance with the following procedure.

The crude peptide was dissolved in water/acetonitrile (65:35) (100 ml) adjusted to pH 7.5 with NH₄OH, and was purified by semipreparative HPLC on a 25 mm×250 mm column packed with 7μ C-18 silica. The column was eluted with a gradient of 50 to 70% acetonitrile against 0.1% TFA/water at 10 ml/min at a temperature of 40° C. for 47 min. The peptide-containing fractions were collected, diluted with 3 volumes of water and lyophilized. This afforded 617 mg (11% yield) of hexadecanoyl-Gly-Thr-D-Gln-His-Thr-Nle-c[Glu-Hyp-D-Phe-Arg-Trp-Lys]-NH₂.

LCMS: Rt=13.95 min (100% purity by UV 214 nm); ((m+2)/2)=868.

Examples of further compounds of the invention which may be obtained in a manner analogous to the compound of Example 1 are the compounds of Examples 2-23, below. The synthesis of the building block employed in the synthesis of the title compounds of Examples 12, 13, 15 and 18-23 is described below:

### Example 2

### Hexadecanoyl-Gly-Ser-D-Gln-His-Ser-Nle-c[Glu-Hyp-D-Phe-Arg-Trp-Lys]-NH₂

### Example 3

### Hexadecanoyl-Gly-Ser-D-Gln-His-Ser-Nle-c[Glu-Dap-D-Phe-Arg-Trp-Lys]-NH₂

### Example 4

### Hexadecanoyl-Gly-Ser-D-Gln-His-Ser-Nle-c[Glu-Cit-D-Phe-Arg-Trp-Lys]-NH₂

### Example 5

### Hexadecanoyl-Gly-Ser-D-Gln-His-Ser-Nle-c[Glu-Asn-D-Phe-Arg-Trp-Lys]-NH₂

### Example 6

### Hexadecanoyl-Gly-Ser-D-Gln-His-Ser-Nle-c[Glu-Gln-D-Phe-Arg-Trp-Lys]-NH₂

### Example 7

### Hexadecanoyl-Gly-Gln-D-Gln-His-Ser-Nle-c[Glu-Hyp-D-Phe-Arg-Trp-Lys]-NH₂

### Example 8

### Hexadecanoyl-Gly-Gln-D-Asn-His-Ser-Nle-c[Glu-Hyp-D-Phe-Arg-Trp-Lys]-NH₂

### Example 9

### Hexadecanoyl-Gly-Ser-D-Asn-His-Ser-Nle-c[Glu-Dap-D-Phe-Arg-Trp-Lys]-NH₂

### Example 10

### Hexadecanoyl-Gly-Ser-D-Asn-His-Ser-Nle-c[Glu-Hyp-D-Phe-Arg-Trp-Lys]-NH₂

### Example 11

### Octadecanoyl-Gly-Ser-D-Gln-His-Ser-Nle-c[Glu-Dap-D-Phe-Arg-Trp-Lys]-NH₂

### Example 12

### 4-(Hexadecanoylsulfamoyl)butanoyl-Gly-Ser-D-Gln-His-Ser-Nle-c[Glu-Hyp-D-Phe-Arg-Trp-Lys]-NH₂

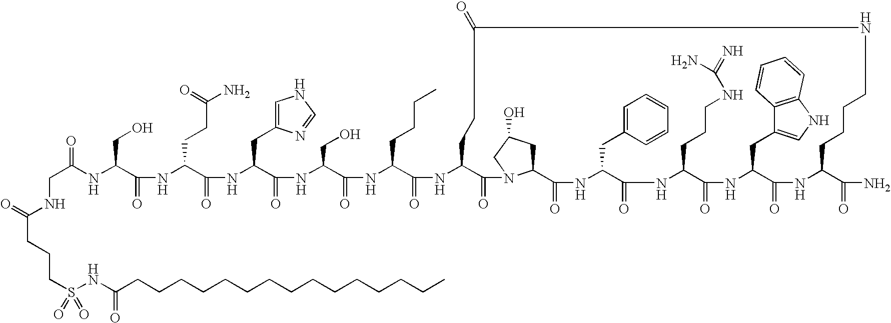

### Example 13

### 4-(Hexadecanoylsulfamoyl)butanoyl-Gly-Ser-D-Asn-His-Ser-Nle-c[Glu-Hyp-D-Phe-Arg-Trp-Lys]-NH₂

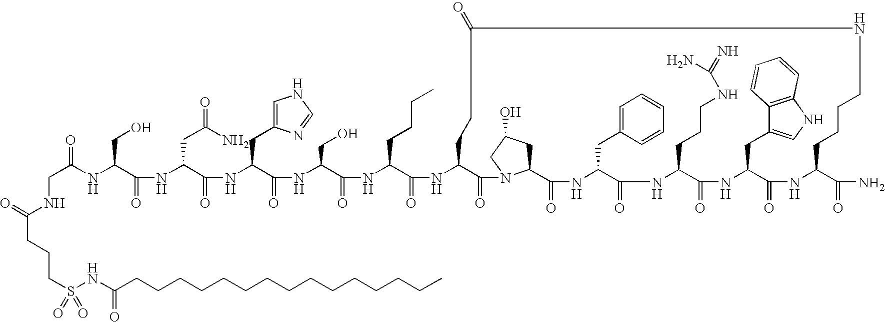

### Example 14

### 2-[2-(Hexadecanoylamino)ethoxy]ethoxyacetyl-Gly-Thr-D-Gln-His-Ser-Nle-c[Glu-Hyp-D-Phe-Arg-Trp-Lys]-NH₂

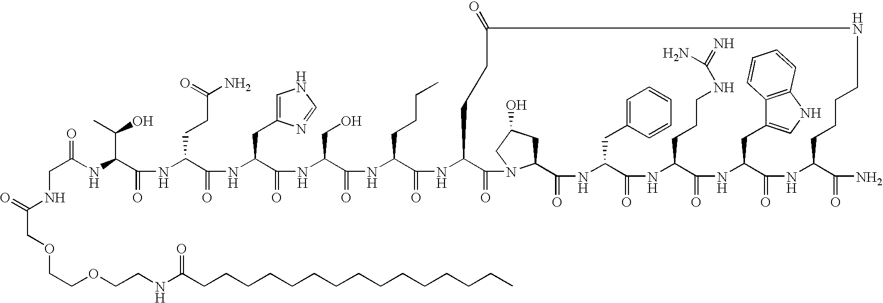

### Example 15

### 4-(Hexadecanoylsulfamoyl)butanoyl-Gly-Thr-D-Gln-His-Thr-Nle-c[Glu-Hyp-D-Phe-Arg-Trp-Lys]-NH₂

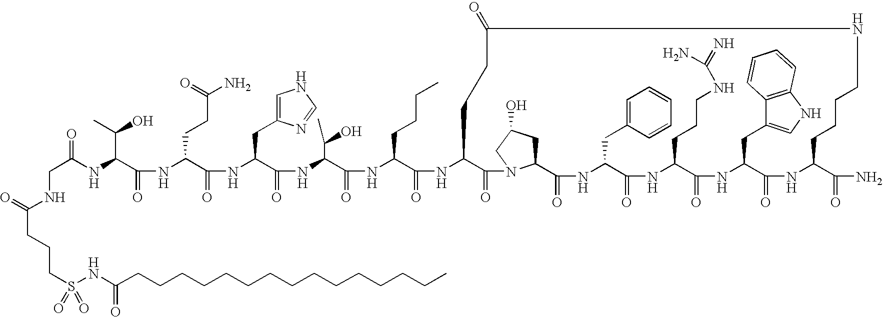

LCMS: Rt=15.39 min; ((m+2)/2)=942.

### Example 16

### 4-(Tridecanoylsulfamoyl)butanoyl-Gly-Thr-D-Gln-His-Thr-Nle-c[Glu-Hyp-D-Phe-Arg-Trp-Lys]-NH₂

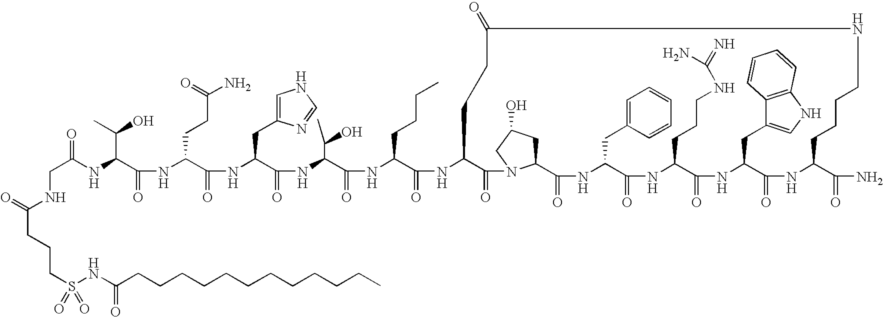

LCMS: Rt=12.80 min; ((m+2)/2)=922.

### Example 17

### Hexadecanoyl-Gly-Ser-D-Gln-His-Ser-Nle-c[homoCys-Hyp-D-Phe-Arg-Trp-homoCys]-NH₂

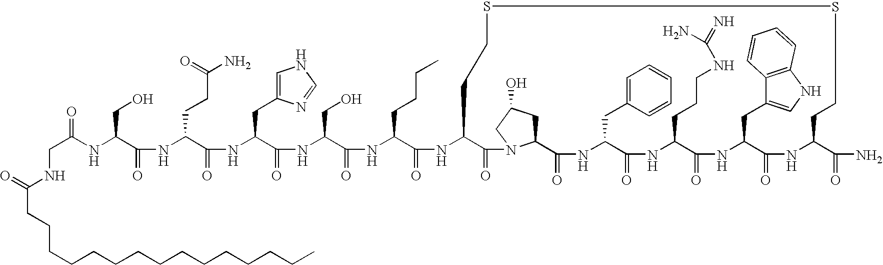

### Example 18

### 4-(Hexadecanoylsulfamoyl)butanoyl-Gly-Ser-D-Gln-His-Dap-Nle-c[Cys-Hyp-D-Phe-Arg-Trp-Pen]-NH₂

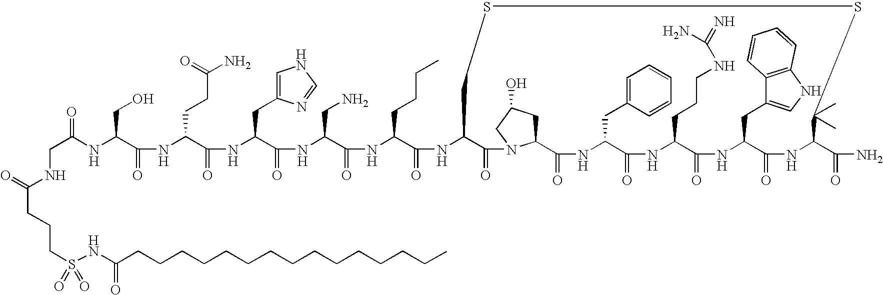

### Example 19

### 4-(Hexadecanoylsulfamoyl)butanoyl-Gly-Ser-D-Gln-His-Dap-Nle-c[Cys-Hyp-D-Phe-Arg-Trp-Cys]-NH₂

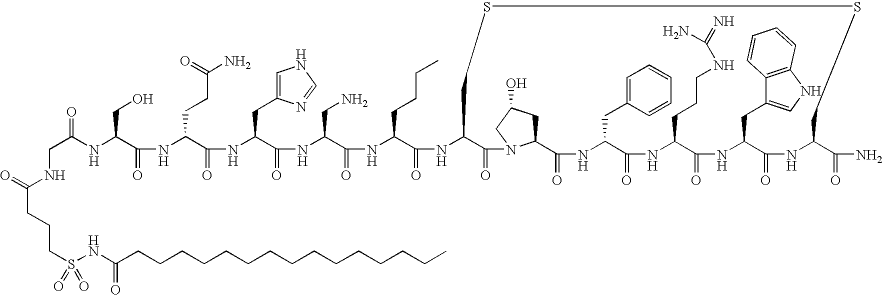

### Example 20

### 4-(Hexadecanoylsulfamoyl)butanoyl-Glu-Ser-D-Gln-His-Ser-Nle-c[Glu-Hyp-D-Phe-Arg-Trp-Lys]-NH₂

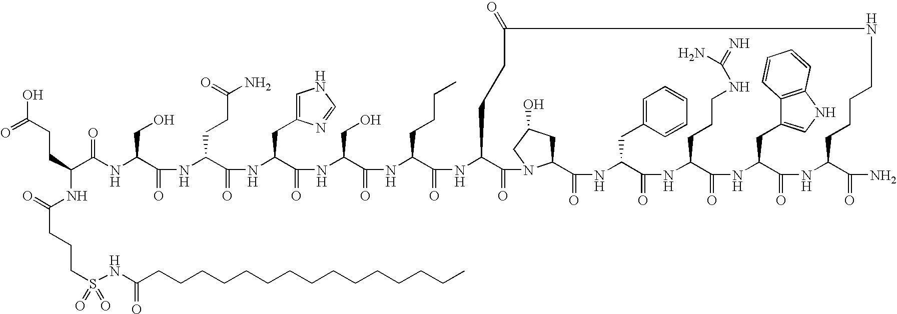

### Example 21

### 4-(Hexadecanoylsulfamoyl)butanoyl-Gly-Glu-D-Gln-His-Ser-Nle-c[Glu-Hyp-D-Phe-Arg-Trp-Lys]-NH₂

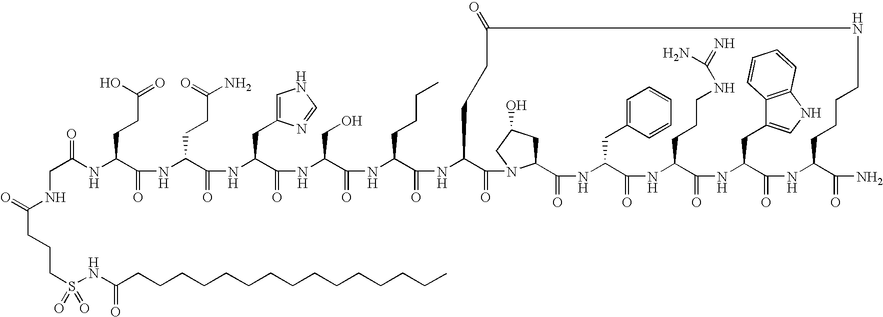

### Example 22

### 4-(Hexadecanoylsulfamoyl)butanoyl-Glu-Ser-D-Gln-His-Glu-Nle-c[Glu-Hyp-D-Phe-Arg-Trp-Lys]-NH₂

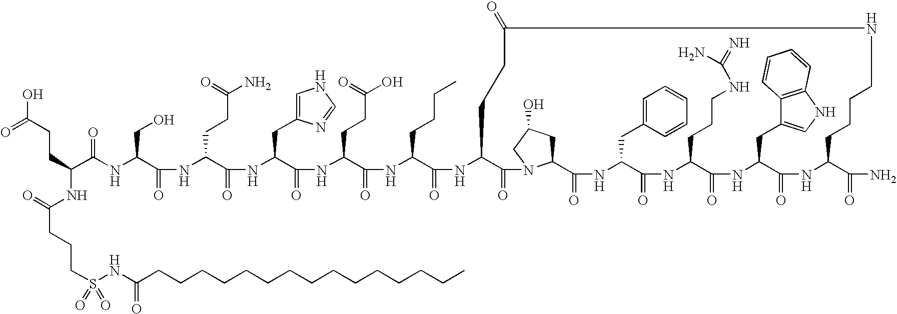

### Example 23

### 4-(Hexadecanoylsulfamoyl)butanoyl-Gly-Ser-D-Gln-His-Ser-Ala-c[Glu-Hyp-D-Phe-Arg-Trp-Lys]-NH₂

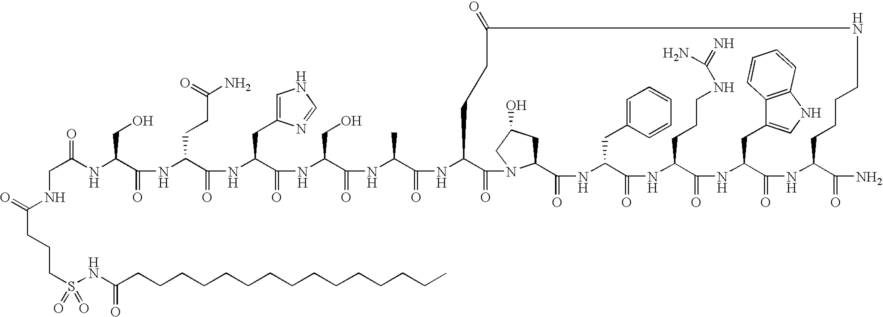

### Preparation of 4-(hexadecanoylsulfamoyl)butyric acid for Use in the Syntheses of the Title Compounds of Examples 12, 13, 15 and 18-23

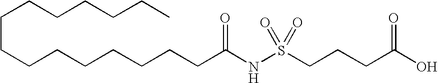

The synthesis of this building block has been described previously in WO 2004/099246 (Novo Nordisk A/S). Briefly, 4-sulfamoylbutyric acid methyl ester is prepared from commercially available 4-sulfamoylbutyric acid and acylated with palmitic chloride in the presence of 4-dimethylaminopyridine. The resulting acylsulfonamide is isolated. Saponification with sodium hydroxide and recrystallisation affords 4-(hexadecanoylsulfamoyl)butyric acid.

### Pharmacological Methods

Assay (I)—Experimental Protocol for Efficacy Testing on Appetite with MC4 Analogues, Using an Ad Libitum Fed Rat Model.

TAC:SPRD rats or Wistar rats from M&B Breeding and Research Centre A/S, Denmark are used for the experiments. The rats have a body weight 200-250 g at the start of experiment. The rats arrive at least 10-14 days before start of experiment with a body weight of 180-200 g. Each dose of compound is tested in a group of 8 rats. A vehicle group of 8 rats is included in each set of testing.

When the animals arrive they are housed individually in a reversed light/dark phase (lights off 7:30 am, lights on 7:30 pm), meaning that lights are off during daytime and on during night-time. Since rats normally initiate food intake when light is removed, and eat the major part of their daily food intake during the night, this set-up results in an alteration of the initiation time for food intake to 7:30 am, when lights are switched off. During the acclimatization period of 10-14 days, the rats have free access to food and water. During this period the animals are handled at least 3 times. The experiment is conducted in the rats' home cages. Immediately before dosing the rats are randomised to the various treatment groups (n=8) by body weight. They are dosed according to body weight at between 7:00 am and 7:45 am, with a 1-3 mg/kg solution administered intraperitoneally (ip), orally (po) or subcutaneously (sc). The time of dosing is recorded for each group. After dosing, the rats are returned to their home cages, where they then have access to food and water. The food consumption is recorded individually every hour for 7 hours, and then after 24 h and sometimes 48 h. At the end of the experimental session, the animals are euthanised.

The individual data are recorded in Microsoft excel sheets. Outliers are excluded after applying the Grubbs statistical evaluation test for outliers, and the result is presented graphically using the GraphPad Prism program.

Assay (II)—Melanocortin Receptor 3 and 5 (MC3 and MC5) cAMP Functional Assay Using the AlphaScreen™ cAMP Detection Kit

The cAMP assays for MC3 and MC5 receptors are performed on cells (either HEK293 or BHK cells) stably expressing the MC3 and MC5 receptors, respectively. The receptors are cloned from cDNA by PCR and inserted into the pcDNA 3 expression vector. Stable clones are selected using 1 mg/ml G418.

Cells at approx. 80-90% confluence are washed 3× with PBS, lifted from the plates with Versene and diluted in PBS. They are then centrifuged for 2 min at 1300 rpm, and the supernatant removed. The cells are washed twice with stimulation buffer, and then resuspended in stimulation buffer to a final concentration of 1×10⁶ or 2×10⁶ cells/ml. 25 μl of cell suspension is added to the microtiter plates containing 25 μl of test compound or reference compound (all diluted in stimulation buffer). The plates are incubated for 30 minutes at room temperature (RT) on a plate-shaker set to a low rate of shaking. The reaction is stopped by adding 25 μl of acceptor beads with anti-cAMP, and 2 min later 50 μl of donor beads per well with biotinylated cAMP in a lysis buffer. The plates are then sealed with plastic, shaken for 30 minutes and allowed to stand overnight, after which they are counted in an Alpha™ microplate reader.

EC₅₀ values are calculated by non-linear regression analysis of dose/response curves (6 points minimum) using the Windows™ program GraphPad™ Prism (GraphPad™ Software, USA). All results are expressed in nM.

For measuring antagonistic activity in the MC3 functional cAMP assay, the MC3 receptors are stimulated with 3 nM α-MSH, and inhibited with increasing amounts of the potential antagonist. The IC₅₀ value for the antagonist is defined as the concentration that inhibits MC3 stimulation by 50%.

Assay (III)—Melanocortin Receptor 4 (MC4) cAMP Assay

BHK cells expressing the MC4 receptor are stimulated with potential MC4 agonists, and the degree of stimulation of cAMP is measured using the Flash Plate® cAMP assay (NEN™ Life Science Products, cat. No. SMP004).

The MC4 receptor-expressing BHK cells are produced by transfecting the cDNA encoding MC4 receptor into BHK570/KZ10-20-48, and selecting for stable clones expressing the MC4 receptor. The MC4 receptor cDNA, as well as a CHO cell line expressing the MC4 receptor, may be purchased from Euroscreen™. The cells are grown in DMEM, 10% FCS, 1 mg/ml G418, 250 nM MTX and 1% penicillin/streptomycin.

Cells at approx. 80-90% confluence are washed 3× with PBS, lifted from the plates with Versene and diluted in PBS. They are then centrifuged for 2 min at 1300 rpm, and the supernatant removed. The cells are washed twice with stimulation buffer, and resuspended in stimulation buffer to a final concentration of 0.75×10⁶ cells/ml (consumption thereof: 7 ml per 96-well microtiter plate). 50 μl of cell suspension is added to the Flash Plate containing 50 μl of test compound or reference compound (all diluted in H₂O). The mixture is shaken for 5 minutes and then allowed to stand for 25 minutes at RT. The reaction is stopped by addition of 100 μl Detection Mix per well (Detection Mix=11 ml Detection Buffer+100 μl (˜2 μCi) cAMP [¹²⁵I] tracer). The plates are then sealed with plastic, shaken for 30 minutes, and allowed to stand overnight (or for 2 hours) and then counted in the Topcounter (2 min/well). In general, the assay procedure is as described in the Flash Plate kit-protocol (Flash Plate® cAMP assay (NEN™ Life Science Products, cat. No. SMP004)). However, the cAMP standards are diluted in 0.1% HSA and 0.005% Tween™ 20 and not in stimulation buffer.

EC₅₀ values are calculated by non-linear regression analysis of dose/response curves (6 points minimum) using the Windows™ program GraphPad™ Prism (GraphPad Software, USA). All results are expressed in nM.

### Assay (IV)—Melanocortin Receptor 1 (MC1) Binding Assay

The MC1 receptor binding assay is performed on BHK cell membranes stably expressing the MC1 receptor. The assay is performed in a total volume of 250 μl: 25 μl of ¹²⁵NDP-α-MSH (22 μM in final concentration), 25 μl of test compound/control and 200 μl of cell membrane (35 μg/ml). Test compounds are dissolved in DMSO. Radioactively labeled ligand, membranes and test compounds are diluted in buffer: 25 mM HEPES, pH 7.4, 0.1 mM CaCl₂, 1 mM MgSO₄, 1 mM EDTA, 0.1% HSA and 0.005% Tween™ 20. The samples are incubated at 30° C. for 90 min in Greiner microtiter plates, separated with GF/B filters that are pre-wetted for 60 min in 0.5% PEI, and washed 2-3 times with NaCl (0.9%) before separation of bound from unbound radiolabelled ligand by filtration. After filtration the filters are washed 10 times with ice-cold 0.9% NaCl. The filters are dried at 50° C. for 30 min, sealed, and 30 μl of Microscint 0 (Packard, cat. No. 6013616) is added to each well. The plates are counted in a Topcounter (1 min/well).

The data are analysed by non-linear regression analysis of binding curves, using the Windows™ program GraphPad™ Prism (GraphPad Software, USA).

### Assay (V)—Melanocortin Receptor 4 (MC4) Binding Assay

### In Vitro ¹²⁵NDP-α-MSH Binding to Recombinant BHK Cells Expressing Human MC4 Receptor (Filtration Assay).

The assay is performed in 5 ml minisorb vials (Sarstedt No. 55.526) or in 96-well filterplates (Millipore MADVN 6550), and using BHK cells expressing the human MC4 receptor (obtained from Professor Wikberg, Uppsala, Sweden). The BHK cells are kept at −80° C. until assay, and the assay is run directly on a dilution of this cell suspension, without further preparation. The suspension is diluted to give maximally 10% specific binding, i.e. to approx. 50-100 fold dilution. The assay is performed in a total volume of 200 μl: 50 μl of cell suspension, 50 μl of ¹²⁵NDP-A-MSH (≈79 μM in final concentration), 50 μl of test compound and 50 μl binding buffer (pH 7) mixed and incubated for 2 h at 25° C. [binding buffer: 25 mM HEPES (pH 7.0), 1 mM CaCl₂, 1 mM MgSO₄, 1 mM EGTA, 0.02% Bacitracin and 0.2% BSA]. Test compounds are dissolved in H₂O and diluted in binding buffer. Radiolabelled ligand and membranes are diluted in binding buffer. The incubation is stopped by dilution with 5 ml ice-cold 0.9% NaCl, followed by rapid filtration through Whatman GF/C filters pre-treated for 1 hour with 0.5% polyethyleneimine. The filters are washed with 3×5 ml ice-cold NaCl. The radioactivity retained on the filters is counted using a Cobra II auto gamma counter.

The data are analysed by non-linear regression analysis of binding curves, using the Windows™ program GraphPad™ Prism (GraphPad Software, USA).

### Assay (VI)—Evaluation of Energy Expenditure

TAC:SPRD rats or Wistar rats from M&B Breeding and Research Centre A/S, Denmark are used. After at least one week of acclimatization, rats are placed individually in metabolic chambers (Oxymax system, Columbus Instruments, Columbus, Ohio, USA; systems calibrated daily). During the measurements, animals have free access to water, but no food is provided to the chambers. Light:dark cycle is 12 h:12 h, with lights being switched on at 6:00. After the animals have spent approx. 2 hours in the chambers (i.e. when the baseline energy expenditure is reached), test compound or vehicle are administered (po, ip or sc), and recording is continued in order to establish the action time of the test compound. Data for each animal (oxygen consumption, carbon dioxide production and flow rate) are collected every 10-18 min for a total of 22 hours (2 hours of adaptation (baseline) and 20 hours of measurement). Correction for changes in O₂ and CO₂ content in the inflowing air is made in each 10-18 min cycle.

Data are calculated per metabolic weight [(kg body weight) 0.75] for oxygen consumption and carbon dioxide production, and per animal for heat. Oxygen consumption (VO₂) is regarded as the major energy expenditure parameter of interest.

1. A compound of formula I:
R¹—S-Z¹-Z²-Z³-His-Z⁴-Z⁵-c[X¹—X²—X³-Arg-X⁴—X⁵]N(R′)₂ [I]
wherein R¹ represents a straight-chain, branched and/or cyclic C₁₄₋₂₂alkanoyl, C₁₄₋₂₂alkenoyl or C₁₄₋₂₂alkynoyl which may optionally be substituted with one or more substituents selected from halogen, hydroxy and aryl, or R¹ represents C₉₋₁₇—C(O)—NH—S(O)₂—(CH₂)₃—C(O)—;
S represents a bond, a 4-aminobutyric acid residue, Gly, β-Ala or a structure represented by formula II 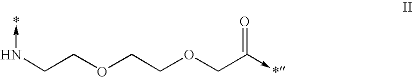
Z¹ represents Gly, β-Ala, Ser, D-Ser, Thr, D-Thr, His, D-His, Asn, D-Asn, Gln, D-Gln, Glu, D-Glu, Asp, D-Asp, Ala or D-Ala;
Z² represents Ser, Thr, Gln, Asn, Glu, Asp or His;
Z³ represents D-Gln or D-Asn;
Z⁴ represents Ser, Thr, Dab, Dap, Glu or Asp;
Z⁵ represents Ala, Val, Leu, Ile, Met or Nle;
X¹ represents Glu, Asp, Cys, homoCys, Pen, Lys, Orn, Dab or Dap;
X² represents His, Cit, Dab, Dap, Cgl, Cha, Val, Ile, tBuGly, Leu, Tyr, Glu, Ala, Nle, Met, Met(O), Met(O₂), Gln, Gln(alkyl), Gln(aryl), Asn, Asn(alkyl), Asn(aryl), Ser, Thr, Cys, Pro, Hyp, Tic, 2-PyAla, 3-PyAla, 4-PyAla, (2-thienyl)alanine, 3-(thienyl)alanine, (4-thiazolyl)Ala, (2-furyl)alanine, (3-furyl)alanine or Phe, wherein the phenyl moiety of said Phe is optionally substituted with a substituent selected among halogen, hydroxy, alkoxy, nitro, benzoyl, methyl, trifluoromethyl, amino and cyano;
X³ represents D-Phe, wherein the phenyl moiety in D-Phe may optionally be substituted with one or more substituents selected among halogen, hydroxy, alkoxy, nitro, methyl, trifluoromethyl and cyano;
X⁴ represents Trp, 2-Nal, a (3-benzo[b]thienyl)alanine residue or a (S)-2,3,4,9-tetrahydro-1H-β-carboline-3-carboxylic acid residue;
X⁵ represents Glu, Asp, Cys, homoCys, Pen, Lys, Orn, Dab or Dap;
wherein X¹ and X⁵ are joined, rendering the compound of formula I cyclic, either via a disulfide bridge deriving from X¹ and X⁵ both independently being Cys, homoCys or Pen, or via an amide bond formed between a carboxylic acid in the side-chain of X¹ and an amino group in the side chain of X⁵, or between a carboxylic acid in the side-chain of X⁵ and an amino group in the side-chain of X¹;
each R′ independently represents hydrogen or C₁₋₆alkyl, which may optionally be substituted with one or more amino or hydroxy;
with the proviso that the compound of formula I is not tetradecanoyl-Gly-Ser-D-Gln-His-Thr-Nle-c[Glu-Hyp-D-Phe-Arg-Trp-Lys]-NH₂, hexadecanoyl-Gly-Ser-D-Gln-His-Thr-Nle-c[Glu-Hyp-D-Phe-Arg-Trp-Lys]-NH₂, octadecanoyl-Gly-Ser-D-Gln-His-Thr-Nle-c[Glu-Hyp-D-Phe-Arg-Trp-Lys]-NH₂ or hexadecanoyl-Gly-Ser-D-Asn-His-Thr-Nle-c[Glu-Hyp-D-Phe-Arg-Trp-Lys]-NH₂;
and pharmaceutically acceptable salts, prodrugs and solvates thereof.

2. A compound according to claim 1, wherein R¹ is C₁₄₋₁₈-alkanoyl, and S is a bond or a structure represented by formula II.

3. A compound according to claim 1, wherein R¹ is 4-(C₁₄₋₁₈alkanoylsulfamoyl)butanoyl, and S is a bond.

4. A compound according to claim 3, wherein R¹ is 4-(hexadecanoylsulfamoyl)butanoyl.

5. A compound according to claim 1, wherein Z¹ is Gly, Glu or Asp, and Z⁵ is Nle or Ala.

6. A compound according to claim 1, wherein Z² is Glu, Asp, Ser, Thr, Gln or Asn.

7. A compound according to claim 6, wherein Z² is Ser, Thr, Gln or Asn.

8. A compound according to claim 7, wherein Z² is Thr or Ser.

9. A compound according to claim 1, wherein Z³ is D-Gln.

10. A compound according to claim 1, wherein Z³ is D-Asn.

11. A compound according to claim 1, wherein Z⁴ is Glu, Asp, Ser, Thr, Dab or Dap.

12. A compound according to claim 11, wherein Z⁴ is Ser or Thr.

13. A compound according to claim 1, wherein Z⁵ is Nle.

14. A compound according to claim 1, wherein Z⁵ is Ala.

15. A compound according to claim 1, wherein X¹ is Glu, X³ is D-Phe, X⁴ is Trp and X⁵ is Lys.

16. A compound according to claim 1, wherein X¹ is Asp, X³ is D-Phe, X⁴ is Trp and X⁵ is Lys.

17. A compound according to claim 1, wherein X¹ and X⁵ independently are Cys, homoCys or Pen, X³ is D-Phe, and X⁴ is Trp.

18. A compound according to claim 1, wherein X² is Tic, Met(O)₂, Ser, Hyp, Cit, Dap, Asn, Gln or (4-thiazolyl)Ala.

19. A compound according to claim 18, wherein X² is Hyp.

20. A compound according to claim 18, wherein X² is Asn or Gln.

21. A compound according to claim 18, wherein X² is Cit.

22. A compound according to claim 1, wherein N(R′)₂ is NH₂.

23. A compound according to claim 1, selected among:
R¹—S-Gly-Ser-D-Gln-His-Ser-Nle-c[Glu-Hyp-D-Phe-Arg-Trp-Lys]-NH₂,
R¹—S-Gly-Thr-D-Gln-His-Ser-Nle-c[Glu-Hyp-D-Phe-Arg-Trp-Lys]-NH₂,
R¹—S-Gly-Gln-D-Gln-His-Ser-Nle-c[Glu-Hyp-D-Phe-Arg-Trp-Lys]-NH₂,
R¹—S-Gly-Ser-D-Asn-His-Ser-Nle-c[Glu-Hyp-D-Phe-Arg-Trp-Lys]-NH₂,
R¹—S-Gly-Thr-D-Asn-His-Ser-Nle-c[Glu-Hyp-D-Phe-Arg-Trp-Lys]-NH₂,
R¹—S-Gly-Gln-D-Asn-His-Ser-Nle-c[Glu-Hyp-D-Phe-Arg-Trp-Lys]-NH₂,
R¹—S-Gly-Ser-D-Gln-His-Thr-Nle-c[Glu-Hyp-D-Phe-Arg-Trp-Lys]-NH₂,
R¹—S-Gly-G-D-Gln-His-Thr-Nle-c[Glu-Hyp-D-Phe-Arg-Trp-Lys]-NH₂,
R¹—S-Gly-Ser-D-Asn-His-Thr-Nle-c[Glu-Hyp-D-Phe-Arg-Trp-Lys]-NH₂
R¹—S-Gly-Thr-D-Asn-His-Thr-Nle-c[Glu-Hyp-D-Phe-Arg-Trp-Lys]-NH₂,
R¹—S-Gly-Gln-D-Asn-His-Thr-Nle-c[Glu-Hyp-D-Phe-Arg-Trp-Lys]-NH₂,
R¹—S-Gly-Ser-D-Gln-His-Ser-Nle-c[Glu-Cit-D-Phe-Arg-Trp-Lys]-NH₂,
R¹—S-Gly-Thr-D-Gln-His-Ser-Nle-c[Glu-Cit-D-Phe-Arg-Trp-Lys]-NH₂,
R¹—S-Gly-Gln-D-Gln-His-Ser-Nle-c[Glu-Cit-D-Phe-Arg-Trp-Lys]-NH₂,
R¹—S-Gly-Ser-D-Asn-His-Ser-Nle-c[Glu-Cit-D-Phe-Arg-Trp-Lys]-NH₂,
R¹—S-Gly-Thr-D-Asn-His-Ser-Nle-c[Glu-Cit-D-Phe-Arg-Trp-Lys]-NH₂,
R¹—S-Gly-Gln-D-Asn-His-Ser-Nle-c[Glu-Cit-D-Phe-Arg-Trp-Lys]-NH₂,
R¹—S-Gly-Ser-D-Gln-His-Thr-Nle-c[Glu-Cit-D-Phe-Arg-Trp-Lys]-NH₂,
R¹—S-Gly-Thr-D-Gln-His-Thr-Nle-c[Glu-Cit-D-Phe-Arg-Trp-Lys]-NH₂,
R¹—S-Gly-Gln-D-Gln-His-Thr-Nle-c[Glu-Cit-D-Phe-Arg-Trp-Lys]-NH₂,
R¹—S-Gly-Ser-D-Asn-His-Thr-Nle-c[Glu-Cit-D-Phe-Arg-Trp-Lys]-NH₂,
R¹—S-Gly-Thr-D-Asn-His-Thr-Nle-c[Glu-Cit-D-Phe-Arg-Trp-Lys]-NH₂,
R¹—S-Gly-Gln-D-Asn-His-Thr-Nle-c[Glu-Cit-D-Phe-Arg-Trp-Lys]-NH₂,
R¹—S-Gly-Ser-D-Gln-His-Ser-Nle-c[Glu-Asn-D-Phe-Arg-Trp-Lys]-NH₂,
R¹—S-Gly-Thr-D-Gln-His-Ser-Nle-c[Glu-Asn-D-Phe-Arg-Trp-Lys]-NH₂,
R¹—S-Gly-Gln-D-Gln-His-Ser-Nle-c[Glu-Asn-D-Phe-Arg-Trp-Lys]-NH₂
R¹—S-Gly-Ser-D-Asn-His-Ser-Nle-c[Glu-Asn-D-Phe-Arg-Trp-Lys]-NH₂,
R¹—S-Gly-Thr-D-Asn-His-Ser-Nle-c[Glu-Asn-D-Phe-Arg-Trp-Lys]-NH₂,
R¹—S-Gly-Gln-D-Asn-His-Ser-Nle-c[Glu-Asn-D-Phe-Arg-Trp-Lys]-NH₂,
R¹—S-Gly-Ser-D-Gln-His-Thr-Nle-c[Glu-Asn-D-Phe-Arg-Trp-Lys]-NH₂,
R¹—S-Gly-Thr-D-Gln-His-Thr-Nle-c[Glu-Asn-D-Phe-Arg-Trp-Lys]-NH
R¹—S-Gly-Gln-D-Gln-His-Thr-Nle-c[Glu-Asn-D-Phe-Arg-Trp-Lys]-NH₂
R¹—S-Gly-Ser-D-Asn-His-Thr-Nle-c[Glu-Asn-D-Phe-Arg-Trp-Lys]-NH₂,
R¹—S-Gly-Thr-D-Asn-His-Thr-Nle-c[Glu-Asn-D-Phe-Arg-Trp-Lys]-NH₂,
R¹—S-Gly-Gln-D-Asn-His-Thr-Nle-c[Glu-Asn-D-Phe-Arg-Trp-Lys]-NH₂,
R¹—S-Gly-Ser-D-Gln-His-Ser-Nle-c[homoCys-Hyp-D-Phe-Arg-Trp-homoCys]-NH₂,
R¹—S-Gly-Ser-D-Gln-His-Dap-Nle-c[Cys-Hyp-D-Phe-Arg-Trp-Pen]-NH₂,
R¹—S-Gly-Ser-D-Gln-His-Dap-Nle-c[Cys-Hyp-D-Phe-Arg-Trp-Cys]-NH₂,
R¹—S-Glu-Ser-D-Gln-His-Ser-Nle-c[Glu-Hyp-D-Phe-Arg-Trp-Lys]-NH₂,
R¹—S-Gly-Glu-D-Gln-His-Ser-Nle-c[Glu-Hyp-D-Phe-Arg-Trp-Lys]-NH₂,
R¹—S-Glu-Ser-D-Gln-His-Glu-Nle-c[Glu-Hyp-D-Phe-Arg-Trp-Lys]-NH₂, and
R¹—S-Gly-Ser-D-Gln-His-Ser-Ala-c[Glu-Hyp-D-Phe-Arg-Trp-Lys]-NH₂.

24. A compound according to claim 1, selected from the group consisting of:
hexadecanoyl-Gly-Thr-D-Gln-His-Thr-Nle-c[Glu-Hyp-D-Phe-Arg-Trp-Lys]-NH₂,
hexadecanoyl-Gly-Ser-D-Gln-His-Ser-Nle-c[Glu-Hyp-D-Phe-Arg-Trp-Lys]-NH₂,
hexadecanoyl-Gly-Ser-D-Gln-His-Ser-Nle-c[Glu-Dap-D-Phe-Arg-Trp-Lys]-NH₂,
hexadecanoyl-Gly-Ser-D-Gln-His-Ser-Nle-c[Glu-Cit-D-Phe-Arg-Trp-Lys]-NH₂,
hexadecanoyl-Gly-Ser-D-Gln-His-Ser-Nle-c[Glu-Asn-D-Phe-Arg-Trp-Lys]-NH₂,
hexadecanoyl-Gly-Ser-D-Gln-His-Ser-Nle-c[Glu-Gln-D-Phe-Arg-Trp-Lys]-NH₂,
hexadecanoyl-Gly-Gln-D-Gln-His-Ser-Nle-c[Glu-Hyp-D-Phe-Arg-Trp-Lys]-NH₂,
hexadecanoyl-Gly-Gln-D-Asn-His-Ser-Nle-c[Glu-Hyp-D-Phe-Arg-Trp-Lys]-NH₂,
hexadecanoyl-Gly-Ser-D-Asn-His-Ser-Nle-c[Glu-Dap-D-Phe-Arg-Trp-Lys]-NH₂,
hexadecanoyl-Gly-Ser-D-Asn-His-Ser-Nle-c[Glu-Hyp-D-Phe-Arg-Trp-Lys]-NH₂,
octadecanoyl-Gly-Ser-D-Gln-His-Ser-Nle-c[Glu-Dap-D-Phe-Arg-Trp-Lys]-NH₂,
4-(hexadecanoylsulfamoyl)butanoyl-Gly-Ser-D-Gln-His-Ser-Nle-c[Glu-Hyp-D-Phe-Arg-Trp-Lys]-NH₂,
4-(hexadecanoylsulfamoyl)butanoyl-Gly-Ser-D-Asn-His-Ser-Nle-c[Glu-Hyp-D-Phe-Arg-Trp-Lys]-NH₂,
2-[2-(hexadecanoylamino)ethoxy]ethoxyacetyl-Gly-Thr-D-Gln-His-Ser-Nle-c[Glu-Hyp-D-Phe-Arg-Trp-Lys]-NH₂,
4-(hexadecanoylsulfamoyl)butanoyl-Gly-Thr-D-Gln-His-Thr-Nle-c[Glu-Hyp-D-Phe-Arg-Trp-Lys]—NH₂,
4-(tridecanoylsulfamoyl)butanoyl-Gly-Thr-D-Gln-His-Thr-Nle-c[Glu-Hyp-D-Phe-Arg-Trp-Lys]-NH₂,
hexadecanoyl-Gly-Ser-D-Gln-His-Ser-Nle-c[homoCys-Hyp-D-Phe-Arg-Trp-homoCys]-NH₂,
4-(hexadecanoylsulfamoyl)butanoyl-Gly-Ser-D-Gln-His-Dap-Nle-c[Cys-Hyp-D-Phe-Arg-Trp-Pen]-NH₂,
4-(hexadecanoylsulfamoyl)butanoyl-Gly-Ser-D-Gln-His-Dap-Nle-c[Cys-Hyp-D-Phe-Arg-Trp-Cys]-NH₂,
4-(hexadecanoylsulfamoyl)butanoyl-Glu-Ser-D-Gln-His-Ser-Nle-c[Glu-Hyp-D-Phe-Arg-Trp-Lys]-NH₂,
4-(hexadecanoylsulfamoyl)butanoyl-Gly-Glu-D-Gln-His-Ser-Nle-c[Glu-Hyp-D-Phe-Arg-Trp-Lys]-NH₂,
4-(hexadecanoylsulfamoyl)butanoyl-Glu-Ser-D-Gln-His-Glu-Nle-c[Glu-Hyp-D-Phe-Arg-Trp-Lys]-NH₂, and
4-(hexadecanoylsulfamoyl)butanoyl-Gly-Ser-D-Gln-His-Ser-Ala-c[Glu-Hyp-D-Phe-Arg-Trp-Lys]-NH₂.

25. A method of delaying the progression from IGT to type 2 diabetes, comprising administering to a patient in need thereof an effective amount of a compound according to claim 1, optionally in combination with one or more additional therapeutically active compounds.

26. A method of delaying the progression from type 2 diabetes to insulin-requiring diabetes, comprising administering to a patient in need thereof an effective amount of a compound according to claim 1, optionally in combination with one or more additional therapeutically active compounds.

27. A method of treating obesity or preventing overweight, comprising administering to a patient in need thereof an effective amount of a compound according to claim 1, optionally in combination with one or more additional therapeutically active compounds.

28. A method of regulating appetite, comprising administering to a patient in need thereof an effective amount of a compound according to claim 1, optionally in combination with one or more additional therapeutically active compounds.

29. A method of inducing satiety, comprising administering to a patient in need thereof an effective amount of a compound according to claim 1, optionally in combination with one or more additional therapeutically active compounds.

30. A method of preventing weight gain after successfully having lost weight, comprising administering to a patient in need thereof an effective amount of a compound according to claim 1, optionally in combination with one or more additional therapeutically active compounds.

31. A method of increasing energy expenditure, comprising administering to a patient in need thereof an effective amount of a compound according to claim 1, optionally in combination with one or more additional therapeutically active compounds.

32. A method of treating a disease or state related to overweight or obesity, comprising administering to a patient in need thereof an effective amount of a compound according to claim 1, optionally in combination with one or more additional therapeutically active compounds.

33. A method of treating bulimia, comprising administering to a patient in need thereof an effective amount of a compound according to claim 1, optionally in combination with one or more additional therapeutically active compounds.

34. A method of treating binge-eating, comprising administering to a patient in need thereof an effective amount of a compound according to claim 1, optionally in combination with one or more additional therapeutically active compounds.

35. A method of treating a disease or state selected from atherosclerosis, hypertension, dia-betes, type 2 diabetes, impaired glucose tolerance (IGT), dyslipidemia, coronary heart dis-ease, gallbladder disease, gall stone, osteoarthritis, cancer, sexual dysfunction and risk of premature death, comprising administering to a patient in need thereof an effective amount of a compound according to claim 1, optionally in combination with one or more additional therapeutically active compounds.

36. A method of treating, in an obese patient, a disease or state selected from type 2 diabetes, impaired glucose tolerance (IGT), dyslipidemia, coronary heart disease, gallbladder disease, gall stone, osteoarthritis, cancer, sexual dysfunction and risk of premature death, comprising administering to an obese patient in need thereof an effective amount of a compound according to claim 1, optionally in combination with one or more additional therapeutically active compounds.

37. A method according to claim 25, wherein said additional therapeutically active compound is selected from antidiabetic agents, antihyperlipidemic agents, antiobesity agents, antihypertensive agents and agents for the treatment of complications resulting from, or associated with, diabetes.

38. A method according to claim 25, wherein said compound is administered to said patient in a unit dosage form comprising from about 0.05 mg to about 1000 mg of said compound.

39. A method of activating MC4 in a subject, the method comprising administering to said subject an effective amount of a compound according to claim 1.

40. A method according to claim 39, wherein said compound is administered parenterally by nasal, pulmonary or sublingual administration.

41. (canceled)

42. A pharmaceutical composition comprising a compound according to claim 1.

43. (canceled)

44. A method according to claim 26, wherein said additional therapeutically active compound is selected from antidiabetic agents, antihyperlipidemic agents, antiobesity agents, antihypertensive agents and agents for the treatment of complications resulting from, or associated with, diabetes.

45. A method according to claim 26, wherein said compound is administered to said patient in a unit dosage form comprising from about 0.05 mg to about 1000 mg of said compound.

46. A method according to claim 27, wherein said additional therapeutically active compound is selected from antidiabetic agents, antihyperlipidemic agents, antiobesity agents, antihypertensive agents and agents for the treatment of complications resulting from, or associated with, diabetes.

47. A method according to claim 27, wherein said compound is administered to said patient in a unit dosage form comprising from about 0.05 mg to about 1000 mg of said compound.

48. A method according to claim 28, wherein said additional therapeutically active compound is selected from antidiabetic agents, antihyperlipidemic agents, antiobesity agents, antihypertensive agents and agents for the treatment of complications resulting from, or associated with, diabetes.

49. A method according to claim 28, wherein said compound is administered to said patient in a unit dosage form comprising from about 0.05 mg to about 1000 mg of said compound.

50. A method according to claim 29, wherein said additional therapeutically active compound is selected from antidiabetic agents, antihyperlipidemic agents, antiobesity agents, antihypertensive agents and agents for the treatment of complications resulting from, or associated with, diabetes.

51. A method according to claim 29, wherein said compound is administered to said patient in a unit dosage form comprising from about 0.05 mg to about 1000 mg of said compound.

52. A method according to claim 30, wherein said additional therapeutically active compound is selected from antidiabetic agents, antihyperlipidemic agents, antiobesity agents, antihypertensive agents and agents for the treatment of complications resulting from, or associated with, diabetes.

53. A method according to claim 30, wherein said compound is administered to said patient in a unit dosage form comprising from about 0.05 mg to about 1000 mg of said compound.

54. A method according to claim 31, wherein said additional therapeutically active compound is selected from antidiabetic agents, antihyperlipidemic agents, antiobesity agents, antihypertensive agents and agents for the treatment of complications resulting from, or associated with, diabetes.

55. A method according to claim 31, wherein said compound is administered to said patient in a unit dosage form comprising from about 0.05 mg to about 1000 mg of said compound.

56. A method according to claim 32, wherein said additional therapeutically active compound is selected from antidiabetic agents, antihyperlipidemic agents, antiobesity agents, antihypertensive agents and agents for the treatment of complications resulting from, or associated with, diabetes.

57. A method according to claim 32, wherein said compound is administered to said patient in a unit dosage form comprising from about 0.05 mg to about 1000 mg of said compound.

58. A method according to claim 33, wherein said additional therapeutically active compound is selected from antidiabetic agents, antihyperlipidemic agents, antiobesity agents, antihypertensive agents and agents for the treatment of complications resulting from, or associated with, diabetes.

59. A method according to claim 33, wherein said compound is administered to said patient in a unit dosage form comprising from about 0.05 mg to about 1000 mg of said compound.

60. A method according to claim 34, wherein said additional therapeutically active compound is selected from antidiabetic agents, antihyperlipidemic agents, antiobesity agents, antihypertensive agents and agents for the treatment of complications resulting from, or associated with, diabetes.

61. A method according to claim 34, wherein said compound is administered to said patient in a unit dosage form comprising from about 0.05 mg to about 1000 mg of said compound.

62. A method according to claim 36, wherein said additional therapeutically active compound is selected from antidiabetic agents, antihyperlipidemic agents, antiobesity agents, antihypertensive agents and agents for the treatment of complications resulting from, or associated with, diabetes.

63. A method according to claim 35, wherein said compound is administered to said patient in a unit dosage form comprising from about 0.05 mg to about 1000 mg of said compound.

64. A method according to claim 36, wherein said additional therapeutically active compound is selected from antidiabetic agents, antihyperlipidemic agents, antiobesity agents, antihypertensive agents and agents for the treatment of complications resulting from, or associated with, diabetes.

65. A method according to claim 36, wherein said compound is administered to said patient in a unit dosage form comprising from about 0.05 mg to about 1000 mg of said compound.
